(19) United States
(12) Patent Application Publication
(10) Pub. No.: US 2008/0139456 A1
(43) Pub. Date: Jun. 12, 2008

(54) Macrocyclic Sh2 Domain Binding Inhibitors

(76) Inventors: Terrence R. Burke, Bethesda, MD (US); Zhen-Dan Shi, Bethesda, MD (US); Shinya Oishi, Kyoto (JP); Fa Liu, Frederick, MD (US)

Correspondence Address: LEYDIG, VOIT & MAYER, LTD., TWO PRUDENTIAL PLAZA, SUITE 4900, 180 NORTH STETSON AVENUE, CHICAGO, IL, 60601-6731, US

(21) Appl. No.: 11/575,723

(22) PCT Filed: Sep. 30, 2005
(86) PCT No.: PCT/US05/35246

§ 371 Date: Mar. 21, 2007

(51) Int. Cl. A61K 38/12; C07K 5/12

(52) U.S. Cl. 514/7; 530/317

(57) ABSTRACT

Disclosed are compounds for inhibiting the binding of an SH2 domain-containing protein, for example, a compound of formula (I): FORMULA (I) wherein R1 is a lipophile; R2, in combination with the phenyl ring, is a phenylphosphate mimic group or a protected phenylphosphate mimic group; R3 is, for example, hydrogen, azido, amino, oxalylamino, carboxy alkyl, alkoxycarbonyl alkyl, aminocarbonyl alkyl, or alkyl carbonylamino; R6 is a linker; AA is an amino acid; and n is 1 to 6; or a pharmaceutically acceptable salt, stereoisomer, solvate, or hydrate thereof. Also disclosed are pharmaceutical compositions and methods of use of such compounds.

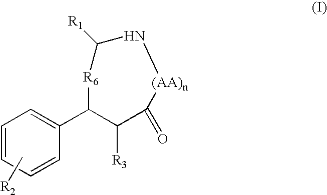

### CROSS-REFERENCE TO RELATED APPLICATION

This application claims the benefit of U.S. Provisional Patent Application No. 60/614,800, filed Sep. 30, 2004, the disclosure of which is incorporated by reference.

### FIELD OF THE INVENTION

This invention relates to macrocyclic peptides, compositions comprising these peptides, and methods of using these peptides, e.g., in inhibiting SH2 domain-containing protein from binding with a phosphoprotein and in the prevention or treatment of a disease such as cancer in a mammal.

### BACKGROUND OF THE INVENTION

The pharmaceutical industry is in search for new classes of compounds for the therapy and prophylaxis of proliferative diseases such as cancer, autoimmune diseases, and hyperproliferative skin disorders such as psoriasis. These diseases or disorders affect a large portion of the population, leading to suffering and possibly death.

Some of these diseases or disorders may involve signal transduction. Signal transduction is critical to normal cellular homeostasis and is the process of relaying extracellular messages, e.g., chemical messages in the form of growth factors, hormones and neurotransmitters, via receptors, e.g., cell-surface receptors, to the interior of the cell. Protein-tyrosine kinases play a central role in this biological function. Among others, these enzymes catalyze the phosphorylation of specific tyrosine residues to form tyrosine phosphorylated residues. Examples of this class of enzymes include the PDGF receptor, the FGF receptor, the HGF receptor, members of the EGF receptor family such as the EGF receptor, erb-B2, erb-B3 and erb-B4, the src kinase family, Fak kinase and the Jak kinase family. The tyrosine-phosphorylated proteins are involved in a range of metabolic processes, from proliferation and growth to differentiation.

Protein-tyrosine phosphorylation is known to be involved in modulating the activity of some target enzymes as well as in generating specific complex networks involved in signal transduction via various proteins containing a specific amino acid sequence called an Src homology region or SH2 domain (see, e.g., Proc. Natl. Acad. Sci. USA, 90, 5891 (1990)). A malfunction in this protein-tyrosine phosphorylation through tyrosine kinase overexpression or deregulation is manifested by various oncogenic and (hyper-) proliferative disorders such as cancer, inflammation, autoimmune disease, hyper-proliferative skin disorders, such as psoriasis, and allergy/asthma. SH2- and/or SH3-comprising proteins that play a role in cellular signaling and transformation include, but are not limited to, the following: Src, Lck, Eps, ras GTPase-activating protein (GAP), phospholipase C, phosphoinositol-3 (PI-3) kinase, Fyn, Lyk, Fgr, Fes, ZAP-70, Sem-5, p85, SHPTP1, SHPTP2, corkscrew, Syk, Lyn, Yes, Hck, Dsrc, Tec, Atk/Bpk, Itk/Tsk, Arg, Csk, tensin, Vav, Emt, Grb2, BCR-Abl, Shc, Nck, Crk, CrkL, Syp, Blk, 113TF, 91TF, Tyk2, especially Src, phospholipase c, phoshoinositol-3 (PI-3) kinase, Grb2, BCR-Abl, Shc, Nck, Crk, CrkL, Syp, Blk, 113TF, 91TF, and Tyk2. A direct link has been established between activated receptor kinases and Ras with the finding that the mammalian Grb2 protein, a 26 kilo Dalton (kD) protein comprising a single SH2 and two SH3 domains bind to proline-rich sequences present in the Sos exchange factor.

The significance of ras-regulatory proteins in human tumors is also highlighted by the critical role of Grb2 in BCR-Abl mediated oncogenesis (J. Exp. Med., 179, 167-175 (1994)). Involved in the binding of SH2 domains with phosphotyrosine (“pTyr”) containing ligands is the interaction of the doubly ionized pTyr phosphate with two invariant arginine residues in a well-formed pocket. These arginine-phosphate interactions are particularly significant to the overall binding, such that high affinity binding is usually lost by removal of the phosphate group.

There exists a need for molecules that have an ability to mimic the structure of the phosphotyrosine peptide binding site, as well as a need for compounds that have the ability to disrupt the interaction between SH2 domains of proteins (e.g., regulatory proteins) for example that of Grb2, and proteins with phosphorylated moieties. There further exists a need for compounds suitable for use in the therapy or prophylaxis of proliferative diseases or conditions, as well as in diagnosis, assays, and testing.

These and other advantages of the present invention will be apparent from the description as set forth below.

### BRIEF SUMMARY OF THE INVENTION

The invention provides, in aspect, compounds of formula (I):

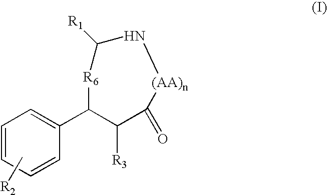

wherein R₁ is a lipophile; R₂, in combination with the phenyl ring, is a phenylphosphate mimic group or a protected phenylphosphate mimic group; R₃ is hydrogen (unsubstituted), azido, amino, oxalylamino, carboxy alkyl, alkoxycarbonyl alkyl, aminocarbonyl alkyl, or alkyl carbonylamino; wherein the alkyl portion of any of the R₃ groups may be optionally substituted with a suitable substituent, for example, one or more selected from the group consisting of halo, hydroxy, carboxyl, amino, amino alkyl, alkyl, alkoxy, and keto, and any combination thereof; R₆ is a linker; AA is an amino acid or fragment thereof; and n is 1 to 6; or a pharmaceutically acceptable salt, stereoisomer, solvate, or hydrate thereof; pharmaceutical compositions thereof, and methods of use thereof.

In another aspect, the invention provides compounds of the formula II:

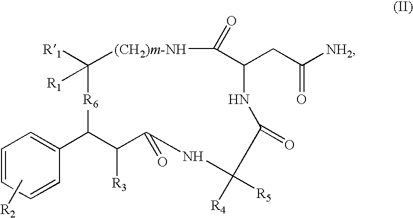

wherein R₁ and R₁′ are the same and are C₁-C₆ alkyl or R₁ and R₁′ together form a C₄-C₈ cycloalkyl; R₂, in combination with the phenyl ring, is a phenylphosphate mimic group or a protected phenylphosphate mimic group; R₃ is hydrogen, azido, amino, oxalylamino, carboxy C₁-C₆ alkyl, C₁-C₆ alkoxycarbonyl C₁-C₆ alkyl, aminocarbonyl C₁-C₆ alkyl, or C₁-C₆ alkyl carbonylamino; wherein the alkyl portion of R₃ may be optionally substituted with a substituent selected from the group consisting of halo, hydroxy, carboxyl, amino, amino C₁-C₆ alkyl, C₁-C₆ alkyl, C₁-C₆ alkoxy, and keto, and any combination thereof; R₄ and R₅, independently, are hydrogen, C₁-C₆ alkyl, C₄-C₈ cycloalkyl, or heterocyclyl, or R₄ and R₅ together form a C₄-C₈ cycloalkyl or heterocyclyl;
R₆ is a group having 1-6 carbon atoms, which may be optionally have a substituent selected from the group consisting of halo, hydroxy, carboxyl, amino, aminoalkyl, C₁-C₆ alkyl, C₁-C₆ alkoxy, and keto, and any combination thereof; and m is 1 or 2;

or a pharmaceutically acceptable salt, stereoisomer, solvate, or hydrate thereof; pharmaceutical compositions thereof, and methods of use thereof.

### BRIEF DESCRIPTION OF THE DRAWINGS

FIG. 1 depicts a method for preparing compounds 3 and 4. Reagents: (i) HOBt, DIPCDI, DMF; (ii) a) TFA; b) aqueous NaHCO₃; c) HOBt, DIPCDI, DMF; (iii) 20% piperidine in DMF; (iv) N-Fmoc-Ac₆c-OH, HOBt, DIPCDI, DMF for 7a; Et₂N/DMF then Boc-Ac₆c-OH, HOAT, EDCI, DMF for 6b; (v) 20% piperidine in DMF; (vi) HOAt, EDCI.HCl, DMF for 8a; TFA-anisole (10:1) for 8b; (vii) [((PCy₃)(Im(Mes)₂)Ru═CHPh)], CH₂Cl₂; (viii) TFA, H₂O.

FIG. 2 depicts the formulas of compounds 3 and 4 in accordance with an embodiment of the invention and of open-chain compound 2.

FIG. 3 depicts a solid-state method for preparing compound 9b, open-chain compound 2, and 15a-15c, 17a-17b, and 18a-18b.

FIG. 4 depicts a reaction scheme to prepare compound 24 in accordance with an embodiment of the invention.

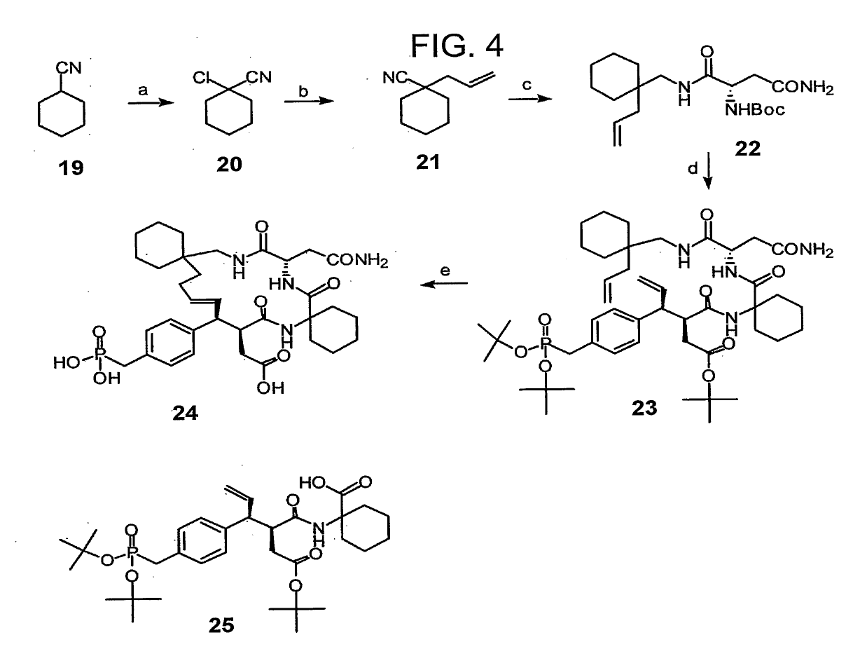

### DETAILED DESCRIPTION OF THE INVENTION

An aspect of the present invention is predicated on the concept that binding affinity for SH2 domain proteins can be envisioned to increase by a conformational constraint in a ligand. The conformational constraint is believed to lead to certain advantages, e.g., a reduction in binding entropy penalty. Binding of natural pTyr-containing ligands to Grb2 SH2 domains takes place in a β-bend fashion, with key interactions occurring in a pTyr binding pocket as well as in a proximal pocket which ligates the amino acid side chain of a pY+2 Asn residue. The present invention provides a novel platform which is expected provide enhanced binding outside the pTyr pocket.

Accordingly, the present invention provides, in an embodiment, compounds of formula (I):

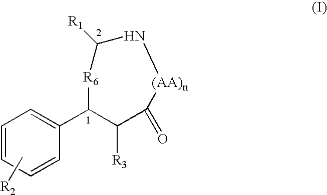

wherein R₁ can be a lipophile; R₂, in combination with the phenyl ring, can be a phenylphosphate mimic group or a protected phenylphosphate mimic group; R₃ can be, for example, hydrogen, azido, amino, oxalylamino, carboxy C₁-C₆ alkyl, C₁-C₆ alkoxycarbonyl C₁-C₆ alkyl, aminocarbonyl C₁-C₆ alkyl, or C₁-C₆ alkyl carbonylamino; wherein the alkyl portion of any of the R₃ groups may be optionally substituted, e.g., with a substituent selected from the group consisting of halo, hydroxy, carboxyl, amino, amino C₁-C₆ alkyl, C₁-C₆ alkyl, C₁-C₆ alkoxy, and keto, and any combination thereof; R₆ is a linker; AA is an amino acid or fragment thereof, and the amino acid can be natural or synthetic; and n can be any suitable integer, e.g., 1 to 6; or a pharmaceutically acceptable salt, stereoisomer, solvate, or hydrate thereof. In an embodiment, n is 2 to 5, preferably 2 or 3, and more preferably 2.

The linker R₆ connects the β-carbon (shown as 1 in formula I) of the pTyr mimetic to the carbon (shown as 2 in formula I) adjacent to the amino group (NH) of the first amino acid. The bond connecting the linker to the linking sites can have any suitable configuration (R, S, or R/S).

R₁ can be any suitable lipophile, e.g., a hydrophobic or nonpolar group, such as an alkyl, alkoxy, alkenyl, alkynyl, aryl, aryloxy, aryl alkoxy, alkylaryl, alkyloxy aryl, arylalkyl, alkylamino, arylalkylamino, alkenylamino, arylamino, aryloxy alkyl, heterocyclyl, heterocyclyloxy, aryl-heterocyclyl alkyl, heterocyclyl alkyl, heterocyclyl alkoxy, aryl heterocyclyl, aryl heterocyclyloxy, alkyl arylalkyl, alkoxy arylalkyl, and alkoxy arylalkoxy, and any combination thereof, optionally substituted or in combination with one or more groups such as alkyl, keto, ester, amino, aminocarbonyl, ureido, hydroxyl, thiol, cyano, alkoxy, and halo. Electron rich groups such as aromatic ring systems such as naphthyl, biphenyl, anthracenyl, and fluorenyl, can be part of examples of R₁ in accordance with an embodiment of the invention. In the various R₁ groups listed above, the alkyl portion can have 1-6 carbon atoms, the aryl portion can have 6-14 carbon atoms, alkenyl and alkynyl portions can have 2-6 carbon atoms, and the heterocyclyl portion can have 3-7 ring atoms including at least one of N, S, and O.

A phenylphosphate mimic group can be one that has the functional property of the phosphorylated-end of tyrosine-phosphorylated sequences, e.g., it can replicate the interaction of phenylphosphate with proteins. The interaction may involve any number of mechanisms, including geometry, size, and/or charge. A protected phenylphosphate mimetic is a phenylphosphate mimic that contains a protecting group that releases the mimetic, e.g., in a biological environment, such as due to chemical or enzymatic hydrolysis. In embodiments, the protecting groups can be esters or amides.

The present invention also provides pharmaceutically acceptable salts of the above compounds including alkali or amine salts. The acidic groups, e.g., carboxylic, phosphoric, or phosphonic groups, of the compound may be converted to salts known to those skilled in the art, for example, a salt of an alkali metal (e.g., sodium or potassium), alkaline earth metal (e.g., calcium), or ammonium salt.

In the above embodiments of the invention, amino acid (AA) may be selected from the group consisting of glycine, alanine, valine, norvaline, leucine, iso-leucine, norleucine, α-amino n-decanoic acid, serine, homoserine, threonine, methionine, cysteine, S-acetylaminomethyl-cysteine, proline, trans-3- and trans-4-hydroxyproline, phenylalanine, tyrosine, 4-aminophenylalanine, 4-nitrophenylalanine, 4-chlorophenylalanine, 4-carboxyphenylalanine, β-phenylserine, β-hydroxyphenylalanine, phenylglycine, α-naphthylalanine, cyclohexylalanine, cyclohexylglycine, tryptophan, indoline-2-carboxylic acid, 1,2,3,4-tetrahydroisoquinoline-3-carboxylic acid, aspartic acid, asparagine, aminomalonic acid, aminomalonic acid monoamide, glutamic acid, glutamine, histidine, arginine, lysine, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid and α,β-diaminopropionic acid, homophenylalanine, and α-tert-butylglycine, and any combination thereof, specifically asparagine and α-aminocyclohexane carboxylic acid.

In accordance with an embodiment, the invention provides compounds of formula (Ia)

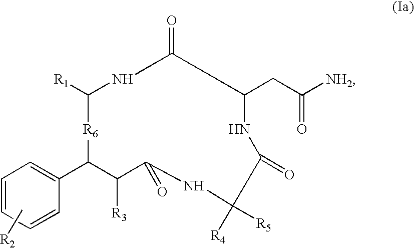

wherein R₄ and R₅, independently, are hydrogen, C₁-C₆ alkyl, C₄-C₈ cycloalkyl, or heterocyclyl, or R₄ and R₅ together form a C₄-C₈ cycloalkyl or a heterocyclyl; R₁-R₃ and R₆ being as described above.

In the embodiments above, examples of R₁ can include C₁-C₆ alkyl carbonyl, C₆-C₁₄ aryl-carbonyl, C₆-C₁₄ aryl C₁-C₆ alkyl carbonyl, C₆-C₁₄ aryl C₁-C₆ alkylamino carbonyl, C₆-C₁₄ aryl C₁-C₆ alkyl, C₆-C₁₄ aryl heterocyclyl C₁-C₆ alkyl, C₆-C₁₄ aryl heterocyclyl C₁-C₆ alkyl carbonyl, C₁-C₆ alkylaminocarbonyl, C₂-C₆ alkenyl aminocarbonyl, C₆-C₁₄ arylaminocarbonyl, C₁-C₆ alkoxy C₁-C₆ alkyl, C₁-C₆ alkoxy C₁-C₆ alkyl carbonyl, C₆-C₁₄ aryloxy C₁-C₆ alkyl, C₆-C₁₄ aryloxy C₁-C₆ alkyl carbonyl, C₆-C₁₄ aryl C₁-C₆ alkoxy C₁-C₆ alkyl, and C₆-C₁₄ aryl C₁-C₆ alkoxy C₁-C₆ alkyl carbonyl, wherein the aryl portion may be optionally substituted or in combination with one or more groups such as alkyl, keto, ester, amino, aminocarbonyl, ureido, hydroxyl, thiol, cyano, alkoxy, and halo.

In the embodiments above, examples of R₂ can include hydroxyl, carboxyl, formyl, carboxy C₁-C₆ alkyl, carboxy C₁-C₆ alkoxy, dicarboxy C₁-C₆ alkyl, dicarboxy C₁-C₆ alkyloxy, dicarboxyhalo C₁-C₆ alkyl, dicarboxyhalo C₁-C₆ alkyloxy, phosphono, phosphono C₁-C₆ alkyl, phosphonohalo C₁-C₆ alkyl, phosphoryl, phosphoryl C₁-C₆ alkyl, and phosphoryl C₁-C₆ alkoxy, carboxy C₁-C₆ alkylamino, oxalylamino, RSO₂NH— wherein R can be C₁-C₆ alkyl, halo C₁-C₆ alkyl, C₆-C₁₄ aryl, C₆-C₁₄ aryl C₁-C₆ alkyl, or trifluoro C₁-C₆ alkyl, C₆-C₁₄ aryl C₁-C₆ alkyl, phosphino C₁-C₆ alkyl, C₁-C₆ alkyl phosphino C₁-C₆ alkyl, C₆-C₁₄ aryl, and C₆-C₁₄ aryl C₁-C₆ alkyl, wherein the alkyl portion of the substituents may be optionally substituted, e.g., with a substituent selected from the group consisting of halo, hydroxy, carboxyl, amino, aminoalkyl, C₁-C₆ alkyl, C₁-C₆ alkoxy, and keto, and any combination thereof.

R₃ in any of the embodiments, can be, for example, hydrogen, azido, amino, oxalylamino, carboxy C₁-C₆ alkyl, C₁-C₆ alkoxycarbonyl C₁-C₆ alkyl, aminocarbonyl C₁-C₆ alkyl, or C₁-C₆ alkyl carbonylamino; wherein the alkyl portion of any of the R₃ groups may be optionally substituted, e.g., with a substituent selected from the group consisting of halo, hydroxy, carboxyl, amino, amino C₁-C₆ alkyl, C₁-C₆ alkyl, C₁-C₆ alkoxy, and keto, and any combination thereof.

R₄ and R₅, independently, are hydrogen, C₁-C₆ alkyl, C₄-C₈ cycloalkyl, or heterocyclyl, or R₄ and R₅ together form a C₄-C₈ cycloalkyl, e.g., cyclohexyl, or a heterocyclyl.

R₆ in any of the embodiments can be a substituted or unsubstituted group having 1-6 carbon atoms, for example, a hydrocarbyl group, such as an unsaturated hydrocarbyl group, optionally further having a substituent selected from the group consisting of halo, hydroxy, carboxyl, amino, aminoalkyl, C₁-C₆ alkyl, C₁-C₆ alkoxy, and keto, and any combination thereof.

In specific embodiments, the invention provides compounds of formulas (Ib)-(Id):

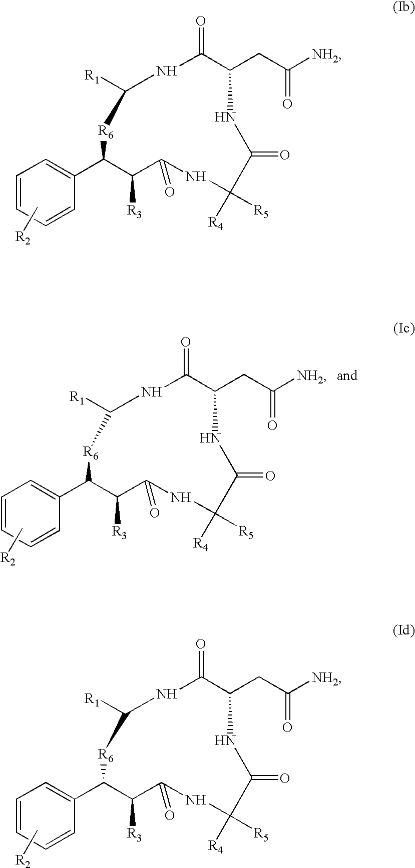

wherein R₁-R₆ can be as described above.

In the embodiments of the invention, specifically, R₁ can be C₁-C₆ alkyl carbonyl, C₆-C₁₄ aryl carbonyl, C₆-C₁₄ aryl C₁-C₆ alkyl carbonyl, C₆-C₁₄ aryl C₁-C₆ alkylamino carbonyl, C₆-C₁₄ aryl heterocyclyl C₁-C₆ alkyl carbonyl, C₁-C₆ alkylaminocarbonyl, C₂-C₆ alkenylaminocarbonyl, C₆-C₁₄ arylamino carbonyl, C₁-C₆ alkoxy C₁-C₆ alkyl carbonyl, C₆-C₁₄ aryloxy C₁-C₆ alkyl carbonyl, or, C₆-C₁₄ aryl C₁-C₆ alkoxy C₁-C₆ alkyl carbonyl, wherein the aryl portion may be unsubstituted or substituted, e.g., with a substituent selected from the group consisting of halo, hydroxy, carboxyl, amino, amino C₁-C₆ alkyl, C₁-C₆ alkyl, C₁-C₆ alkoxy, and keto, and any combination thereof.

In the embodiments of the invention, R₂ can be hydroxyl, carboxyl, formyl, carboxy C₁-C₆ alkyl, carboxy C₁-C₆ alkoxy, dicarboxy C₁-C₆ alkyl, dicarboxy C₁-C₆ alkyloxy, dicarboxyhalo C₁-C₆ alkyl, dicarboxyhalo C₁-C₆ alkyloxy, phosphono, phosphono C₁-C₆ alkyl, phosphonohalo C₁-C₆ alkyl, phosphoryl, phosphoryl C₁-C₆ alkyl, or phosphoryl C₁-C₆ alkoxy, wherein the alkyl or alkoxy portion may be optionally substituted with a substituent, e.g., selected from the group consisting of halo, hydroxy, carboxyl, amino, amino C₁-C₆ alkyl, C₁-C₆ alkyl, C₁-C₆ alkoxy, and keto, and any combination thereof.

In the embodiments of the invention, R₃ can be hydrogen (unsubstituted), azido, amino, oxalylamino, carboxy C₁-C₆ alkyl, C₁-C₆ alkoxycarbonyl C₁-C₆ alkyl, aminocarbonyl C₁-C₆ alkyl, and C₁-C₆ alkylcarbonylamino; wherein the alkyl portion of R₃ may be optionally substituted with a substituent, e.g., selected from the group consisting of halo, hydroxy, carboxyl, amino, amino C₁-C₆ alkyl, C₁-C₆ alkyl, C₁-C₆ alkoxy, and keto, and any combination thereof.

In the embodiments of the invention, R₄ and R₅, independently, can be hydrogen, C₁-C₆ alkyl, cycloalkyl, heterocyclyl, or together form cycloalkyl or heterocyclyl, wherein the cycloalkyl can be a C₃-C₇ cycloalkyl, and the heterocyclyl can be a 3-7 membered ring with at least one of N, O, and S.

In the embodiments of the invention, R₆ can be a C₂-C₆ alkenylenyl or alkynylenyl group, specifically alkenylenyl, any of which may be optionally substituted, e.g., with a substituent selected from the group consisting of halo, hydroxy, carboxyl, amino, amino C₁-C₆ alkyl, C₁-C₆ alkyl, C₁-C₆ alkoxy, and keto, and any combination thereof.

In a specific embodiment, R₁-R₆ are together as described in paragraphs [0027]-[0031].

In the embodiments of the invention, R₁ can be C₆-C₁₄ aryl C₁-C₆ alkylamino carbonyl, for example, C₁₀ aryl C₁-C₆ alkylamino carbonyl, such as naphthyl methylamino carbonyl, e.g., 1-naphthyl methylamino.

In the embodiments of the invention, a specific example of R₂ can be phosphono C₁-C₆ alkyl, optionally substituted with a substituent, e.g., selected from the group consisting of halo, hydroxy, carboxyl, amino, aminoalkyl, alkyl, alkoxy, and keto, and any combination thereof; particularly, R₂ can be phosphonomethyl.

In the embodiments of the invention, R₃ can be carboxy C₁-C₆ alkyl, e.g., carboxymethyl.

In the embodiments of the invention, R₆ can be allyl.

The present invention also provides compounds of the formula II:

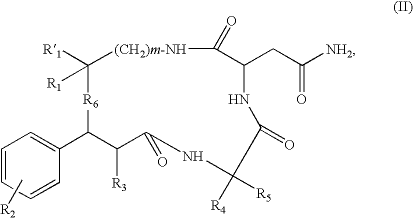

wherein R₁ and R₁′ are the same and are C₁-C₆ alkyl or R₁ and R₁′ together form a C₄-C₈ cycloalkyl; R₂, in combination with the phenyl ring, is a phenylphosphate mimic group or a protected phenylphosphate mimic group; R₃ is hydrogen, azido, amino, oxalylamino, carboxy C₁-C₆ alkyl, C₁-C₆ alkoxycarbonyl C₁-C₆ alkyl, aminocarbonyl C₁-C₆ alkyl, or C₁-C₆ alkyl carbonylamino; wherein the alkyl portion of R₃ may be optionally substituted with a substituent selected from the group consisting of halo, hydroxy, carboxyl, amino, amino C₁-C₆ alkyl, C₁-C₆ alkyl, C₁-C₆ alkoxy, and keto, and any combination thereof; R₄ and R₅, independently, are hydrogen, C₁-C₆ alkyl, C₄-C₈ cycloalkyl, or heterocyclyl, or R₄ and R₅ together form a C₄-C₈ cycloalkyl or heterocyclyl;
R₆ is a group having 1-6 carbon atoms, which may be optionally have a substituent selected from the group consisting of halo, hydroxy, carboxyl, amino, aminoalkyl, C₁-C₆ alkyl, C₁-C₆ alkoxy, and keto, and any combination thereof; and m is 1 or 2;
or a pharmaceutically acceptable salt, stereoisomer, solvate, or hydrate thereof; and specifically, compounds wherein m is 1.

In an embodiment of the compounds of formula II, R₁ and R₁′ together form a C₄-C₈ cycloalkyl, e.g., cyclohexyl.

In any of the embodiments of the compounds of formula II, R₂ is hydroxyl, carboxyl, formyl, carboxy C₁-C₆ alkyl, carboxy C₁-C₆ alkoxy, dicarboxy C₁-C₆ alkyl, dicarboxy C₁-C₆ alkyloxy, dicarboxyhalo C₁-C₆ alkyl, dicarboxyhalo C₁-C₆ alkyloxy, phosphono, phosphono C₁-C₆ alkyl, phosphonohalo C₁-C₆ alkyl, phosphoryl, phosphoryl C₁-C₆ alkyl, and phosphoryl C₁-C₆ alkoxy, carboxy C₁-C₆ alkylamino, oxalylamino, RSO₂NH— wherein R can be C₁-C₆ alkyl, halo C₁-C₆ alkyl, C₆-C₁₄ aryl, C₆-C₁₄ aryl C₁-C₆ alkyl, or trifluoro C₁-C₆ alkyl, C₆-C₁₄ aryl C₁-C₆ alkyl, phosphino C₁-C₆ alkyl, C₁-C₆ alkyl phosphino C₁-C₆ alkyl, C₆-C₁₄ aryl, and C₆-C₁₄ aryl C₁-C₆ alkyl, wherein the alkyl and alkoxy portions of R₂ may be optionally substituted with a substituent selected from the group consisting of halo, hydroxy, carboxyl, amino, aminoalkyl, C₁-C₆ alkyl, C₁-C₆ alkoxy, and keto; and any combination thereof; specifically, R₂ is hydroxyl, carboxyl, formyl, carboxy C₁-C₆ alkyl, carboxy C₁-C₆ alkoxy, dicarboxy C₁-C₆ alkyl, dicarboxy C₁-C₆ alkyloxy, dicarboxyhalo C₁-C₆ alkyl, dicarboxyhalo C₁-C₆ alkyloxy, phosphono, phosphono C₁-C₆ alkyl, phosphonohalo C₁-C₆ alkyl, phosphoryl, phosphoryl C₁-C₆ alkyl, or phosphoryl C₁-C₆ alkoxy, wherein the alkyl and alkoxy portions may be optionally substituted with a substituent selected from the group consisting of halo, hydroxy, carboxyl, amino, amino C₁-C₆ alkyl, C₁-C₆ alkyl, C₁-C₆ alkoxy, and keto, and any combination thereof. In a particular embodiment, R₂ is hydroxyl, carboxyl, formyl, carboxy C₁-C₆ alkyl, carboxy C₁-C₆ alkoxy, dicarboxy C₁-C₆ alkyl, dicarboxy C₁-C₆ alkyloxy, dicarboxyhalo C₁-C₆ alkyl, dicarboxyhalo C₁-C₆ alkyloxy, phosphono, phosphono C₁-C₆ alkyl, phosphonohalo C₁-C₆ alkyl, phosphoryl, phosphoryl C₁-C₆ alkyl, or phosphoryl C₁-C₆ alkoxy. A particular example of R₂ is phosphonomethyl.

In any of the embodiments of the compounds of formula II, R₄ and R₅ together form a C₄-C₈ cycloalkyl, particularly cyclohexyl.

In any of the embodiments of the compounds of formula II, R₆ is a C₂-C₆ alkenylenyl or C₂-C₆ alkynylenyl group, which may optionally have a substituent selected from the group consisting of halo, hydroxy, carboxyl, amino, aminoalkyl, C₁-C₆ alkyl, C₁-C₆ alkoxy, and keto, and any combination thereof.

In any of the embodiments of the compounds of formula II, R₃ is carboxy C₁-C₆ alkyl, e.g., carboxy methyl.

A specific example of the compound of formula IIa is:

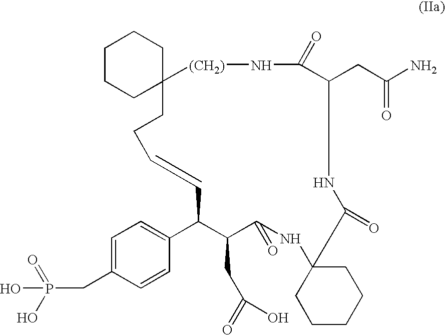

or a pharmaceutically acceptable salt, solvate, or hydrate thereof.

The term “aryl” as used herein refers to an aromatic moiety such as phenyl, naphthyl, anthracenyl, and biphenyl. The term “heterocyclyl” refers to a 3-7 membered ring comprising one or more heteroatoms such as O, N, and S, and optionally carbon and/or hydrogen. Examples of heterocyclyl groups include pyridyl, piperidinyl, piperazinyl, pyrazinyl, pyrolyl, pyronyl, pyronyl, tetrahydropyranyl, tetrahydrothiopyranyl, pyrrolidinyl, furanyl, tetrahydrofuranyl, thiophenyl, tetrahydrothiophenyl, purinyl, pyrimidinyl, thiazolyl, thiazolidinyl, thiazolinyl, oxazolyl, tetrazolyl, tetrazinyl, morpholinyl, thiophorpholinyl, quinolinyl, and isoquinolinyl. The aryl and heterocyclyl moieties may be fused, as in aryl heterocyclyl, such as, e.g., indole, isoindole, benzimidazole, quinoline, isoquinolinyl, benzoxazolyl, benzofuranyl, isobenzofuranyl, carbazolyl, benzodioxolyl, and the like. The alkyl, alkoxy, or alkylamino groups can be linear or branched. When an aryl group is substituted with a substituent, e.g., halo, amino, alkyl, hydroxy, or alkoxy, the aromatic ring hydrogen is replaced with the substituent and this can take place in any of the available atoms (e.g., carbon atoms) bearing a hydrogen, e.g., 2, 3, 4, 5, 6, 7, 8, 9, or 10-position, taking, for the purpose of illustration, the 1-position as the point of attachment of the aryl group. The term “halo” refers to fluorine, chlorine, bromine, or iodine.

The present invention further provides a composition comprising a pharmaceutically acceptable carrier and an effective (e.g., therapeutically or prophylactically effective) amount of at least one of the compounds described above. The present invention further provides a method of inhibiting an SH2 domain from binding with a phosphoprotein comprising contacting a sample or substance containing an SH2 domain with a compound of the present invention.

The present invention discloses the use of above compounds in the manufacture of a medicament for the treatment of a condition that responds to the inhibition of phosphoprotein binding to an SH2 domain of a mammal. The present invention further provides the use of the above compounds in medicine. The compounds can find use as an SH2 domain binding inhibitor. Examples of SH2 domain-containing proteins are Grb2, Shp2, and STAT3 proteins.

The pharmaceutically acceptable (e.g., pharmacologically acceptable) carriers described herein, for example, vehicles, adjuvants, excipients, or diluents, are well known to those who are skilled in the art and are readily available to the publlic. It is preferred that the pharmaceutically acceptable carrier be one which is chemically inert to the active compounds and one which has no detrimental side effects or toxicity under the conditions of use.

The choice of carrier will be determined in part by the particular active agent, as well as by the particular method used to administer the composition. Accordingly, there are a wide variety of suitable formulations of the pharmaceutical composition of the present invention. The following formulations for oral, aerosol, parenteral, subcutaneous, intravenous, intraarterial, intramuscular, interperitoneal, intrathecal, rectal, and vaginal administration are merely exemplary and are in no way limiting.

Formulations suitable for oral administration can comprise (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations can include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art.

The compounds of the present invention, alone or in combination with other suitable components can be made into aerosol formulations to be administered via inhalation. These aerosol formulations can be placed into pressurized acceptable propellants, such as dichlorodifluoromethane, propane, nitrogen, and the like. They also can be formulated as pharmaceuticals for non-pressured preparations, such as in a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The compound can be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

Oils, which can be used in parenteral formulations include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters. Suitable soaps for use in parenteral formulations include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene polypropylene copolymers, (d) amphoteric detergents such as, for example, alkyl-β-aminopropionates, and 2-alkyl-imidazoline quaternary ammonium salts, and (e) mixtures thereof.

The parenteral formulations will typically contain from about 0.5 to about 25% by weight of the active ingredient in solution. Suitable preservatives and buffers can be used in such formulations. In order to minimize or eliminate irritation at the site of injection, such compositions may contain one or more nonionic surfactants. The quantity of surfactant in such formulations typically ranges from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described.

The compounds of the present invention may be made into injectable formulations. The requirements for effective pharmaceutical carriers for injectable compositions are well known to those of ordinary skill in the art; see, e.g., Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4^{th} ed., pages 622-630 (1986).

Additionally, the compounds of the present invention may be made into suppositories by mixing with a variety of bases, such as emulsifying bases or water-soluble bases. Formulations suitable for vaginal administration may be presented as pessaries, tampons, creams, gels, pastes, foams, or spray formulas containing, in addition to the active ingredient, such carriers as are known in the art to be appropriate. Suitable doses and dosage regimens can be determined by conventional range-finding techniques known to those of ordinary skill in the art. Generally, treatment is initiated with smaller dosages, which are less than the optimum dose of the compound. Thereafter, the dosage is increased by small increments until the optimum effect under the circumstances is reached. For convenience, the total daily dosage may be divided and administered in portions during the day if desired. In proper doses and with suitable administration of certain compounds, the present invention provides for a wide range of responses. Typically the dosages range from about 0.001 to about 1000 mg/kg body weight of the animal being treated/day. Preferred dosages range from about 0.01 to about 10 mg/kg body weight/day, and further preferred dosages range from about 0.01 to about 1 mg/kg body weight/day.

Embodiments of the compounds have the advantage that they are stable to or in presence of enzymes encountered during in vivo use. Embodiments of the compounds can find use in in vitro and in vivo applications. For example, the compounds can find use as molecular probes as well as in assays to identify, isolate, and/or quantitate receptor or binding sites in a cell or tissue. The compounds also can find use in vivo for studying the efficacy in the treatment of various diseases or conditions involving SH2 domains.

The present invention further provides a method of preventing or treating a disease, state, or condition in a mammal by the use of the compounds of the present invention. In an embodiment, the method involves preventing a disease, state, or condition. In another embodiment, the method involves treating an existing disease, state, or condition.

In an embodiment, the method involves inhibition of SH2 domain binding with a phosphoprotein. The SH2 domain may involve one or more of the following proteins: Shp2, STAT3, Src, Lck, Eps, ras GTPase-activating protein (GAP), phospholipase C, PI-3 kinase, Fyn, Lyk, Fgr, Fes, ZAP-70, Sem-5, p85, SHPTP1, SHPTP2, corkscrew, Syk, Lyn, Yes, Hck, Dsrc, Tec, Atk/Bpk, Itk/Tsk, Arg, Csk, tensin, Vav, Emt, Grb2, BCR-Abl, Shc, Nck, Crk, CrkL, Syp, Blk, 113TF, 91TF, and Tyk2, especially Grb2, Shp2, and STAT3.

Grb2 is an adaptor protein with N- and C-terminal src homology 3 (SH3) domains and a central src homology 2 (SH2) domain. The SH2 domain can bind to phosphoTyr residues of receptors or other adaptor proteins, such as SHC. The SH3 domains bind the Ras exchange factor SOS, but can also bind to other adaptor proteins such as GAB1 and GAB2. Thus, Grb2 is involved in activation of Ras but can also play a role in other signaling pathways in mammalian cells.

Shp2 is a tyrosine phosphatase that is recruited into tyrosine kinase signaling pathways through binding of its two amino-terminal SH2 domains to specific phosphotyrosine motifs. Shp2 is a member of the protein tyrosine phosphatase (PTP) family. PTPs are known to be signaling molecules that regulate a variety of cellular processes including cell growth, differentiation, mitotic cycle, and oncogenic transformation. Shp2 contains two tandem Src homology-2 domains, which function as phosphotyrosine binding domains and mediate the interaction with its substrates. Shp2 is widely expressed in most tissues and plays a regulatory role in various cell-signaling events that are important for a diversity of cell functions, such as mitogenic activation, metabolic control, transcription regulation, and cell migration.

Signal Transducers and Activators of Transcription (STATs) are transcription factors that are phosphorylated by JAK kinases in response to cytokine activation of a cell surface receptor tyrosine kinases. Upon activation, the STATs dimerize and are localized to the nucleus where they activate transcription of cytokine-responsive genes. There are at least three JAK kinases and at least six STAT proteins involved in this complex signaling pathway. Cytokines that activate STAT3 include growth hormone, IL-6 family cytokines, and G-CSF. STAT3 induces progression through the cell cycle, prevents apoptosis and upregulates oncogenes, such as c-myc and bcl-X and may play a role in oncogenesis. STAT3 has been shown to play a critical role in hematopoiesis. The importance of STAT3 is underscored by the failure of mice lacking STAT3 to survive embryogenesis. Crosstalk from pathways other than JAK kinases also leads to phosphorylation and activation of STAT3 as indicated by a role of mTOR (mammalian target of rapamycin, or p70 S6 kinase) and MAP kinase pathways in STAT3 activation and signaling.

The method of treatment or prevention of a diseases comprises administering to the mammal one or more compounds of the present invention. The disease, state, condition can be a cancer, e.g., a breast cancer or an ovarian cancer, or a tumor such as a solid tumor, e.g., a brain tumor, a prostate tumor, and the like, leukemia including chronic myelocytic leukemia, lymphoma, an autoimmune disease, an inflammatory disease, a metabolic disease, diabetes, obesity, or cardiovascular disease.

The present invention further provides a method of enhancing the therapeutic effect of a treatment rendered to a mammal comprising administering a compound in conjunction with the treatment. By conjunction, it is meant that the inhibitor can be used in any suitable manner, for example, prior to, simultaneous with, or post-administration of the therapeutic agent. Synergistic effects are observed when the SH2 domain binding inhibitor is used in combination with other treatments known to those skilled in the art. The inhibitor enhances the cytotoxicity of the chemotherapeutic treatments. Cancer treatment is particularly suitable for this combination treatment.

The cancer may involve any number of mechanisms. A majority of human breast cancers are dependent upon activation of the Ras signaling pathways through activation of growth factor receptor as the means to achieve continuous cellular proliferation. For example, the cancer may involve overexpression of Her-2/neu. The cancer can be mediated through BCR-Abl or the expression of erbB-2 receptor. In cells transformed by p185 erbB-2 overexpression, therapeutic agents affecting Grb2 function at its SH2 domain may interrupt the flow of signal transduction to the Ras pathway and thus result in reversal of the cancer phenotype.

The therapeutic treatment can include chemotherapy, radiation therapy, and/or a biological therapy. Examples of chemotherapy include the use of cancer treatment agents such as alkylating agents, hormonal agents, antimetabolites, natural products, and miscellaneous agents. Particular examples of cancer treatment agents include paclitaxel, 5-fluoruracil, and doxorubicin. Examples of biological therapy include the use of a protein such as an antibody (monoclonal or polyclonal) or a recombinant protein. An example of an antibody is herceptin, which is targeted for inhibiting the erbB-2 receptor. In embodiments, the enhancement of the therapeutic effect comprises blocking of a cell survival factor in the mammal and/or triggering, e.g., enhancing or speeding up, of cell apoptosis. The treatment can be carried out in vivo and/or in vitro.

The Grb2 SH2 binding inhibitors are effective in inhibiting the association or binding of Grb2 with activated receptor PTKs. Interaction of native Grb2 protein with phosphotyrosinylated proteins including receptor PTKs can be monitored by immunoprecipitating Grb2 and detecting the amount of phosphotyrosinylated proteins which are coprecipitated using anti-phosphotyrosine Western Blotting.

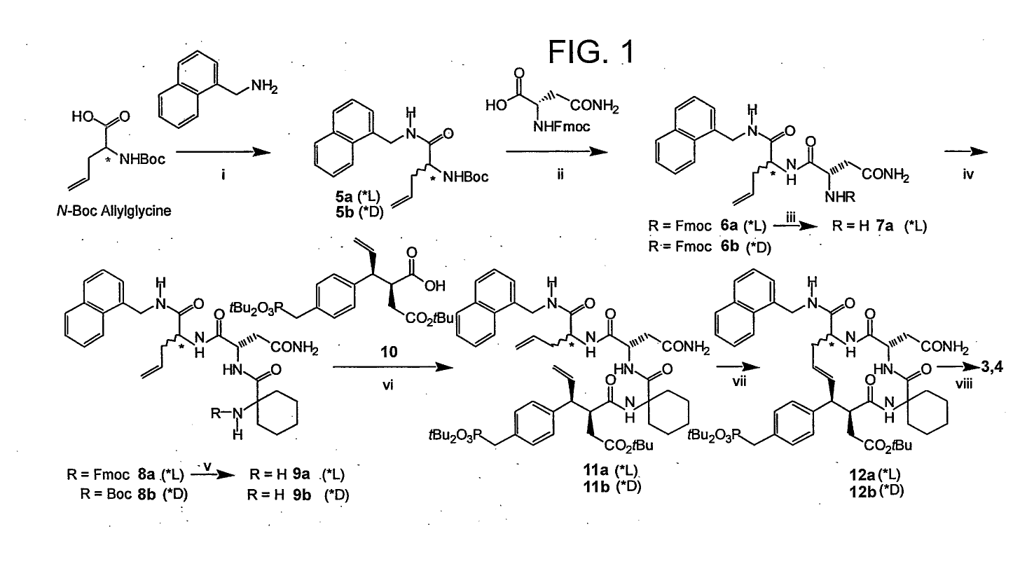

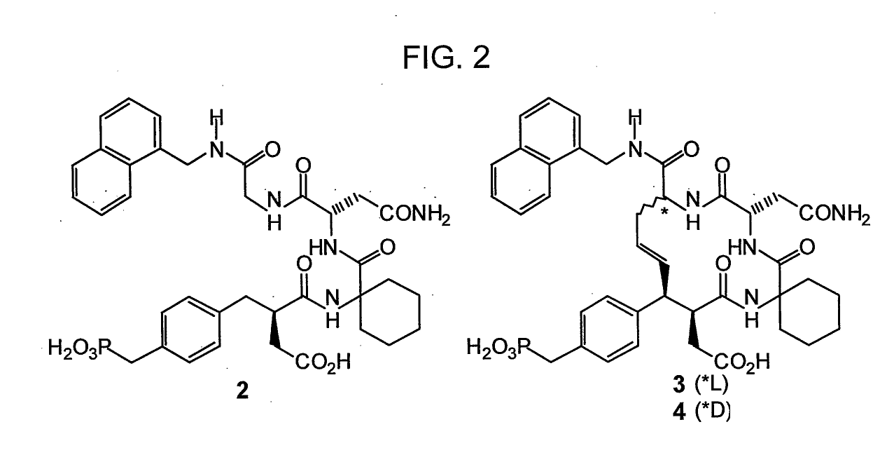

The compounds of the present invention can be prepared by any suitable method, for example, a method that advantageously utilizes C-terminal allylglycine amides, for example, compound 1 in FIG. 1 in a method involving ring closing metathesis (RCM) reaction of allylglycines onto a β-vinyl-containing residue; see, e.g., FIGS. 1-3. For examples of RCM reactions, see, Gao et al., Org. Lett. 2001, 3, 1617-1620; Reichwein et al., Angew. Chem., Int. Ed. 1999, 38, 3684-3687, J. Org. Chem., 2000, 65, 6187-6195; and J. Org. Chem., 2000, 65, 2335-2344; Stymiest et al., Org. Lett., 2003, 5, 47-49; Miller et al., J. Am. Chem. Soc., 1995, 117, 5855-5856; and Dekker et al., Org. Biomol. Chem., 2003, 1, 3297-3303. The RCM reaction advantageously allows ring closure with retention of desired functional groups, e.g., phenylphosphate functionality or the chemical (e.g., a lipophile comprising a carbonyl group) functionality at or near the site of ring juncture(s). L- and D-allylglycines and many of the required terminal amines may be commercially available, thereby making the synthesis more readily accessible than certain other methods, for example, those involving the use of C-terminal 2-allyl-3-aryl-1-propanamides that lacked the α-carboxyl portion of allylglycine residues. In addition to solution chemistries, the preparation of multiple analogues may be made possible through the use of solid-phase chemistries.

The following examples further illustrate the invention but, of course, should not be construed as in any way limiting its scope.

### EXAMPLE 1

This example demonstrates a method of preparing compounds in accordance with an embodiment of the invention.

Reactions were carried out under argon. Anhydrous solvents were purchased from Aldrich Chemical Corporation and used without further drying. Combustion analyses were obtained from Atlantic Microlab, Inc., Norcross, Ga. ¹H NMR spectra were obtained using a Varian 400 MHz spectrometer and are reported in ppm relative to TMS and referenced to the solvent in which they were run. Fast atom bombardment mass spectra (FABMS) were acquired with a VG analytical 7070E mass spectrometer. HPLC separations were conducted using a Waters Prep LC4000 system with photodiode array detection and either a J-sphere ODS-H80 column (20×250 mm) with a solvent system consisting of 0.1% aqueous TFA (v/v, solvent A)/0.1% TFA in MeCN (v/v, solvent B).

N-Boc-L-AllylGly-(1-naphthyl)methyl amide (5a). An ice-cold solution of N-Boc L-allylglycine.dicyclohexylamine salt (Fluka) (1.61 g, 4.05 mmol) in 0.1 N HCl (100 mL) was extracted with ice-cold EtOAc (3×50 mL) and the combined extracts were dried (Na₂SO₄) and taken to dryness to yield the free amine as a syrup (898 mg). To a solution of this syrup in anhydrous DMF (9 mL) was added 1-hydroxybenzotriazole.H₂O(HOBt) (601 mg, 4.46 mmol) and 1,3-diisopropylcarbodiimide (DIPCDI) (696 μL, 4.46 mmol) and after 5 minutes, (1-naphthyl)methylamine (653 μL, 4.46 mmol) and the resulting mixture was stirred at room temperature overnight. The mixture was taken to dryness under high vacuum at 50° C. and the resulting syrup was dissolved in EtOAc and purified by silica gel flash chromatography (hexanes in EtOAc) to yield product 5a as cream-colored crystals (1.15 g, 80% yield). H NMR (CDCl₃) δ 1.31 (s, 9H), 2.44-2.53 (m, 2H), 4.08-4.16 (m, 1H), 4.81-4.94 (m, 2H), 5.04-5.12 (m, 2H), 5.64-5.78 (m, 1H), 6.32-6.38 (br m, 1H), 7.39 (dd, J=7.0 and 10.7 Hz, 1H), 7.46 (dd, J=1.5 and 6.8 Hz, 1H), 7.49 (dd, J=1.3 and 2.1 Hz, 1H), 7.59 (dd, J=1.6 and 7.8 Hz, 1H), 7.77-7.80 (br-m, 1H), 7.83-7.86 (br-m, 1H), 7.94 (br-d, J=8.0 Hz, 1H). FAMBS m/z 355.2 (MH⁺).

N-Boc-D-AllylGly-(1-naphthyl)methyl amide (5b). Treatment of N-Boc-D-allylglycine.dicyclohexylamine salt (PepTech) (2.72 g. 6.86 mmol) in a manner similar to that used to prepare 5a, provided product 5b as a light yellow solid in quantitative yield. H NMR: refer to that provided for enantiomeric 5a. FABMS m/z 355.1 (MH⁺).

N-Fmoc-L-Asn-L-AllylGly-(1-naphthyl)methyl amide (6a). A solution of 5a (254 mg, 1.00 mmol) in TFA: H₂O (9:1) (10 mL) was stirred at room temperature (1 h), then TFA was removed by rotary evaporation under reduced pressure and the residue was partitioned between saturated NaHCO₃ in brine (25 mL) and EtOAc (2×25 mL). The organic extract was dried (Na₂SO₄), evaporated to an oil (203 mg), taken up in DMF (1 mL) and to this was added an HOBt active ester solution prepared by reacting N-Fmoc-L-asparagine (Novabiochem) (357 mg, 1.10 mmol), HOBt (148 mg, 1.10 mmol) and DIPCIDI (172 μL, 1.10 mmol) in DMF (3 mL), 15 minutes. The resulting cleat solution was stirred at room temperature to rapidly yield a thick white suspension. The suspension was diluted with DMF (4 mL) and stirring was continued at room temperature (overnight). Solvent was removed at 50° C. under high vacuum and the residue was triturated well with MeOH (10 mL), collected by filtration and washed well with MeOH to yield product 6a as a snow-white solid (424 mg, 72% yield). H NMR (DMSO-d₆) δ 2.24-2.56 (m, 4H), 4.14-4.20 (m, 3H), 4.24-3.36 (m, 2 HO, 4.66 (d, J=6.9 Hz, 2H), 4.89 (br-d, J=10.4 Hz, 1H), 4.98 (br-d, J=17.2 Hz, 1H), 5.61-5.72 (m, 1H), 6.90 (br-s, 1H), 7.24-7.40 (m, 8H), 7.44-7.50 (m, 2H), 7.57 (d, J=8.0 Hz, 1H), 7.64 (d, J=5.8 Hz, 2H), 7.78-7.79 (m, 1H), 7.84 (d, J=7.5 Hz, 2H), 7.87-7.90 (m, 1H), 8.00 (br-t, J, J=6.1 Hz, 2H), 8.46 (br-t, J=5.6 Hz, 1H). FABMS m/z 591.3 (MH)⁺.

N-Fmoc-L-Asn-D-AllylGly-(1-naphthyl)methyl amide (6b). Treatment of 5b (1.64 g, 6.45 mmol) in a manner similar to that used to prepare 6a from 5a, provided product 6b as white foam in quantitative yield. H NMR (DMSO-d₆) δ 2.24-2.43 (m, 4H), 4.06-4.14 (m, 3H), 4.25-4.34 (m, 2H), 4.65 (d, J=5.8 Hz, 2H), 4.90 (br-d, J=9.6 Hz, 1H), 4.98 (br-d, J=17.5 Hz, 1H), 5.58-5.70 (m, 1H), 6.89 (br-s, 1H), 7.24 (dd, J=7.4 and 15.0 Hz, 2H), 7.30-7.38 (m, 5H), 7.44-7.48 (m, 2H), 7.56 (br-d, J=7.6 Hz, 1H), 7.60 (br-t, J=6.5 Hz, 2H), 7.75 (br-d, J=8.0 Hz, 1H), 7.82-7.88 (m, 3H), 7.96-7.99 (m, 1H), 8.05 (d, J=8.4 8.4 Hz, 1H). FABMS m/z 591.3 (MH)⁺.

H-L-Asn-L-AllylGly-(1-naphthyl)methyl amide (7a). To a suspension of 6a (1.11 g, 1.88 mmol) in DMF (10 mL) was added piperidine (15 equivalents) and the mixture was stirred at room temperature (2 h). Solvent and piperidine were removed under high vacuum to yield a white solid, which was dissolved in MeOH:EtOAc and purified by silica gel flash chromatography (MeOH:EtOAc) to yield free amine 6a as a gel that became a white solid on prolonged exposure to vacuum (540 mg, 78% yield). H NMR (DMSO-d₆) δ 2.12-2.46 (m, 4H), 3.44 (dd, J=5.7 and 8.6 Hz, 1H), 4.30 (br-s, 1H), 4.63-4.53 (m, 2H), 4:95 (dd, J=2.1 and 10.1 Hz, 1H), 5.01 (dd, J=2.1 and 17.2 Hz, 1H), 5.61-5.72 (m, 1H), 6.82 (br-s, 1H), 7.34-7.44 (m, 3H), 7.46-7.52 (m, 3H), 7.79 (br-d, J=7.6H, 1H), 7.87-7.92 (m, 1H), 7.98-8.02 (m, 1H), 8.11 (br-s, 1H), 8.52 (t, J=5.9 Hz, 1H). FABMS m/z 369.2 (MH)⁺.

N-Fmoc-Ac₆c-L-Asn-L-AllylGly-(1-naphthyl)methyl amide (8a). To a solution of amine 7a (368 mg, 1.00 mmol) in DMF (3 mL) was added an HOBt active ester solution prepared by reacting N-Fmoc-1-aminocyclohexane carboxylic acid (N-Fmoc-Ac₆c-OH) (Advanced ChemTech) (420 mg, 1.10 mmol), HOBt (148 mg, 1.10 mmol) and DIPCIDI (172 μL, 1.10 mmol) in DMF (3 mL), 10 minutes and the resulting solution was stirred at room temperature (overnight). The reaction mixture was taken to dryness under high vacuum and purified by silica gel flash chromatography (MeOH:EtOAc) to yield product 8a as white foam in quantitative yield. ¹H NMR (CDCl₃) δ 1.10-1.82 (m, 10H), 2.38-2.56 (m, 2H), 2.73-2.79 (m, 2H), 4.02-4.23 (m, 3H), 4.35-4.45 (m, 2H), 4.62 (dd, J=4.7 and 14.8 Hz, 1H), 4.87 (dd, J=1.7 and 10.3 Hz, 1H), 4.94-5.01 (m, 2H), 5.58-5.75 (m, 1H), 6.82 (s, 1H), 7.18-7.39 (m, 9H), 7.40-7.42 (m, 1H), 7.46-7.48 (m, 1H), 7.61-7.64 (m, 2H), 7.68-7.74 (m, 3H), 7.92 (d, J=8.6 Hz, 1H), 7.96 (s, 1H), 8.27 (d, J=5.7 Hz, 1H). FABMS m/z 716.2 (MH)⁺.

N-Boc-Ac₆c-L-Asn-D-AllylGly-(1-naphthyl)methyl amide (8b). Protected peptide 6b (300 mg, 0.507 mmol) was treated with 20% Et₂NH in DMF (5 mL) for 30 minutes at room temperature. Concentration under reduced pressure gave the corresponding crude amine. To a stirred solution of the amine in dry DMF (1 mL) and 1-hydroxy-7-azabenzotriazole (HOAt) in DMF (0.5 M, 1.11 mL, 0.558 mmol) were added N-Boc-Ac₆c-OH (135 mg, 0.558 mmol) and 1-(3-dimethylaminopropyl)-3-ethylcarbodiimide.HCl (EDCI.HCl) (116 mg, 0.609 mmol), and the mixture was stirred for 12 h at room temperature. The mixture was extracted with EtOAc, and the extract was washed successively with 5% citric acid solution, brine, 5% aqueous NaHCO₃ and brine, and dried over Na₂SO₄. Concentration followed by flash chromatography over silica gel using CH₂Cl₂-MeOH (95:5) provided product 8b as colorless solid (84 mg, 28% yield). ¹H-NMR (400 MHz, CDCl₃) δ 1.25 (m, 2H), 1.40 (s, 9H), 1.63 (m, 6H), 1.84 (m, 1H), 2.03 (m, 1H), 2.39 (dd, J=16.5 and 4.2 Hz, 1H), 2.54 (ddd, J=14.6, 10.7 and 7.7 Hz, 1H), 2.88 (m, 1H), 2.94 (dd, J=16.5 and 4.9 Hz, 1H), 4.32 (br, 1H), 4.45 (m, 1H), 4.58 (m, 2H), 4.90 (s, 1H), 5.05 (m, 2H), 5.15 (dd, J=16.9 and 1.6 Hz, 1H), 5.35 (br, 1H), 5.79 (m, 1H), 7.16 (m, 1H), 7.41 (m, 2H), 7.49 (m, 2H), 7.68 (d, J=8.8 Hz; 1H), 7.74 (m, 2H), 7.81 (d, J=7.9 Hz, 1H), 8.00 (d, J=8.6 Hz, 1H). FABMS m/z 594 (MH)⁺.

H—Ac₆c-L-Asn-L-AllylGly-(1-naphthyl)methyl amide (9a). To a solution of 8a (738 mg, 1.00 mmol) in DMF (5 mL) was added piperidine (1.0 mL, 10 mmol) and the solution was stirred at room temperature (40 minutes). Solvent was then removed under high vacuum at 50° C. and the resulting residue was purified by silica gel flash chromatography (MeOH:EtOAc) to provide an oil. Lyophilization from dioxane provided product 9a as a white solid (343 mg; 84% yield). FABMS m/z 494.3 (MH⁺).

H-AC₆c-L-Asn-D-AllylGly-(1-naphthyl)methyl amide (9b). Preparation of peptide 9b by solid-phase methods is outlined below.

[(2R,3R)-3-(4-Di-tert-butyloxyphosphonomethyl)phenyl-2-(tert-butyloxycarbonylmethyl)pent-4-enyl]-AC₆c-L-Asn-L-AllylGly-(1-naphthyl)methyl amide (11a). To a stirred solution of 9a (70 mg, 0.141 mmol) in dry DMF (0.30 mL) and HOAt in DMF (0.5 M, 0.311 mL, 0.155 mmol) were added protected pTyr mimetic 10 (77 mg, 0.155 mmol; Wei et al., J. Med. Cizem. 2003, 46, 244-254) and EDCI.HCl (32 mg, 0.170 mmol), and the mixture was stirred for 12 h at 50° C. The mixture was extracted with EtOAc, and the extract was washed successively with saturated citric acid solution, brine, 5% aqueous NaHCO₃ solution and brine, and dried over Na₂SO₄. Concentration followed by flash chromatography over silica gel using CH₂Cl₂:MeOH (95:5) provided 11a as colorless solid (16 mg, 12% yield). ¹H-NMR (400 MHz, CDCl₃) δ 0.72-1.62 (m, 37H), 2.47 (m, 2H), 2.62 (m, 3H), 2.82 (m, 1H), 2.96 (d, J=21.5 Hz, 2H), 3.02 (m, 1H), 3.51 (m, 1H), 4.31 (m, 1H), 4.44 (m, 1H), 4.83 (dd, J=15.0 and 5.3 Hz, 1H), 4.97 (dd, J=15.0 and 6.0 Hz, 1H), 5.05 (m, 3H), 5.15 (dd, J=17.1 and 1.6 Hz, 1H), 5.25 (br, 1H), 5.69 (m, 1H), 5.81 (m, 1H), 6.22 (s, 1H), 6.71 (br, 1H), 7.11-7.25 (m, 5H), 7.35-7.55 (m, 5H), 7.59 (d, J=8.3 Hz, 1H), 7.72 (d, J=8.1 Hz, 1H), 7.81 (d, J=9.2 Hz, 1H), 8.04 (d, J=8.1 Hz, 1H). FABMS m/z 972 (MH)⁺.

[(2R,3R)-3-(4-Di-tert-butyloxyphosphonomethyl)phenyl-2-(tert-butyloxycarbonylmethyl)pent-4-enyl]-AC₆c-L-Asn-D-AllylGly-(1-naphthyl)methyl amine (11b). Protected peptide 8b (70 mg, 0.117 mmol) was treated with TFA:anisole (10:1, 5.5 mL) for 2 h at room temperature. Concentration under reduced pressure gave the corresponding amine 9b as its TFA salt. To a stirred solution of the amine 9b in dry DMF (0.300 mL) and HOAt in DMF (0.5 M, 0.259 mL, 0.129 mmol) were added protected pTyr mimetic 10 (64 mg, 0.129 mmol), EDCI.HCl (27 mg, 0.141 mmol) and i-Pr₂NEt (0.040 mL, 0.234 mmol) at 0° C., and stirring was continued for 24 h at 50° C. The mixture was extracted with EtOAc, and the extract was washed successively with saturated citric acid solution, brine, saturated aqueous NaHCO₃ solution and brine, and dried over Na₂SO₄. Concentration followed by flash chromatography over silica gel using CH₂Cl₂:MeOH (95:5) provided 11b as colorless solid (52 mg, 45% yield). ¹H-NMR (400 MHz, CDCl₃) δ 0.78-1.66 (m, 37H), 2.51-2.73 (m, 4H), 2.80 (m, 1H), 2.88-3.05 (m, 4H), 3.32 (t, J=10.0 Hz, 1H), 4.11 (m, 1H), 4.52 (m, 1H), 4.77 (dd, J=15.5 and 5.1 Hz, 1H), 4.99-5.23 (m, 6H), 5.70-5.99 (m, 4H), 7.06 (m, 2H), 7.20 (m, 2H), 7.37 (m, 1H), 7.43-7.61 (m, 6H), 7.72 (d, J=8.1 Hz, 1H), 7.82 (m, 1H), 8.04 (d, J=8.3 Hz, 1H). FABMS m/z 972 (MH)⁺.

Cyclo{[(2R,3R)-3-(4-di-tert-butyloxyphosphonomethyl)phenyl-2-(tert-butyloxycarbonylmethyl)pent-4-enyl]-AC₆c-L-Asn-L-AllylGly}-(1-naphthyl)methyl amide (12a). To a solution of 11a (15 mg, 0.015 mmol) in CH₂Cl₂ (3.7 mL) was added 2nd generation Grubbs RCM catalyst, [1,3-bis-(2,4,6-trimethylphenyl)-2-imidazolidinylidene)dichloro(phenylmethylene)-(tricyclohexylphosphine)ruthenium][((PCy₃)(Im(Mes)₂)Ru═CHPh)] (Aldrich) (6.5 mg, 0.0077 mmol) in CH₂Cl₂ (1.3 mL) under argon. The reaction mixture was stirred at 45° C. for 24 h. The crude reaction mixture was evaporated in vacuo and the residue was purified by silica gel chromatography using CH₂Cl₂:MeOH (10:1) to provide 12a as colorless powder (11 mg, 73% yield). ¹H-NMR (400 MHz, CDCl₃) δ 1.16-1.58 (m, 30H), 1.61 (m, 1H), 1.72 (m, 1H), 1.89 (m, 4H), 2.07 (m, 1H), 2.35 (m, 1H), 2.46-2.73 (m, 4H), 2.99 (m, 3H), 3.57 (m, 1H), 3.88 (m, 1H), 4.83 (m, 2H), 4.90 (m, 1H), 5.13 (m, 2H), 5.83 (dd, J=15.3 and 7.6 Hz, 1H), 6.24 (br, 1H), 7.08 (m, 1H), 7.11-7.25 (m, 4H), 7.34-7.56 (m, 4H), 7.68 (m, 1H), 7.73 (d, J=8.3 Hz, 1H), 7.84 (d, J=7.8 Hz, 1H), 7.98 (d, J=8.8 Hz, 1H), 8.03 (d, J=8.3 Hz, 1H). FABMS m/z 944 (MH)⁺.

Cyclo{[(2R,3R)-3-(4-di-tert-butyloxyphosphonomethyl)phenyl-2-(tert-butyloxycarbonylmethyl)pent-4-enyl]-AC₆c-L-Asn-D-AllylGly}-(1-naphthyl)methyl amide (12b). To a solution of 11b (55 mg, 0.057 mmol) in CH₂Cl₂ (25 mL) was added [((PCy₃)(Im(Mes)₂)Ru═CHPh)] (27 mg, 0.032 mmol) in CH₂Cl₂ (8 mL) under argon and the reaction mixture was stirred at 45° C. (48 h). The crude reaction mixture was then evaporated in vacuo, and residue was purified by silica gel flash chromatography to provide 12b as yellow oil (28 mg, 52% yield). ¹H NMR (CDCl₃) δ 8.04 (m, 1H), 7.87 (d, 1H, J=8.5 Hz), 7.83 (m, 1H), 7.75 (d, 1H, J=8.3 Hz), 7.54-7.40 (m, 6H), 7.24 (dd, 2H, J=2.4 Hz & 8.3 Hz), 7.14 (d, 2H, J=7.9 Hz), 6.81 (d, 1H, J=7.5 Hz), 5.98 (s, 1H), 5.81 (dd, 1H, J=9.1 Hz & 15.5 Hz), 5.72 (s, 1H), 5.40 (m, 1H), 5.02 (m, 1H), 4.79 (dd, 1H, J=5.0 Hz & 14.9 Hz), 4.57 (m, 1H), 4.48 (m, 1H), 3.54 (m, 1H), 3.06 (m, 1H), 3.02 (d, 2H, J=21.5 Hz), 2.97 (m, 1H), 2.88 (dd, 1H, J=5.6 Hz & 15.9 Hz), 2.63 (dd, 1H, J=4.1 Hz & 16.1 Hz), 2.43 (dd, 1H, J=11.2 Hz & 17.2 Hz), 2.23 (m, 1H), 1.95 (dd, 1H, J=3.0 Hz & 17.2 Hz), 1.86-1.44 (m, 6H), 1.42 (s, 9H), 1.40 (s, 9H), 1.36 (s, 9H), 1.31-1.20 (m, 4H). FABMS m/z 944 (MH)⁺.

Cyclo{[(2R,3R)-3-(4-dihydroxyphosphonomethyl)phenyl-2-(hydroxycarbonylmethyl)pent-4-enyl]-AC₆c-L-Asn-L-AlylGly}-1-naphthyl)methyl amide (3). Protected peptide 12a (10 mg, 0.010 mmol) was treated with TFA: H₂O (95:5, 2 mL) for 2 h at room temperature. Concentration followed by preparative HPLC purification (linear gradient 30 to 40% B in A over 30 minutes) provided 3 as colorless powder (4.6 mg, 56% yield). ¹H-NMR (400 MHz, DMSO-d₆) δ 1.23 (m, 1H), 1.48 (m, 5H), 1.78 (m, 4H), 2.06 (m, 1H), 2.35 (m, 1H), 2.42-2.72 (m, 4H), 2.92 (d, J=21.2 Hz, 2H), 3.21 (m, 1H), 4.02 (m, 1H), 4.34 (m, 1H), 4.58 (m, 1H), 4.72 (dd, J=1.5.4 and 5.2 Hz, 1H), 4.81 (dd, J=15.6 and 5.6 Hz, 1H), 5.34 (m, 1. H), 5.84 (dd, J=15.2 and 8.0 Hz, 1H), 6.86 (br, 1H), 7.06 (d, J=8.0 Hz, 1H), 7.18-7.29 (m, 4H), 7.36 (br, 1H), 7.42-7.60 (m, 4H) 7.70 (d, J=8.5 Hz, 1H), 7.83 (d, J=7.2 Hz, 1H), 7.94 (d, J=7.2 Hz, 1H), 8.06 (d, J=8.5 Hz, 1H), 8.16 (s, 1H), 8.31 (t, J=5.4 Hz, 1H). FABMS m/z 774 [(M-H)⁻].

Cyclo{[(2R,3R)-3-(4-dihydroxyphosphonomethyl)phenyl-2-(hydroxycarbonylmethyl)pent-4-enyl]-AC₆c-L-Asn-D-AllylGly}-(1-naphthyl)methyl amide (4). A solution of 12b (22 mg, 0.023 mmol) in a mixture of TFA: triethylsilane (TES): H₂O (4.0 mL, v:v, 3.7:0.1:0.2) was stirred at room temperature (1 h). Solvent was evaporated in vacuo and residue was purified by HPLC using a linear gradient using a linear gradient (5% to 95% B over 15 minutes) provided product 4 as white solid (8.4 mg, 46% yield). ¹H NMR (DMSO-d₆) δ 8.22 (m, 1H), 8.15 (s, 1H), 8.09 (m, 1H), 8.00 (m, 1H), 7.84 (dd, 1H, J=2.6 Hz & 6.7 Hz), 7.72 (d, 1H, J=6.4 Hz), 7.46-7.36 (m, 4H), 7.31 (s, 1H), 7.12-7.06 (m, 5H), 6.83 (s, 1H), 5.80 (dd, 1H, J=9.2 Hz & 15.9 Hz), 5.27 (m, 1H), 4.72-4.64 (m, 2H), 4.56 (m, 1H), 3.91 (m, 1H), 3.81 (m, 1H), 3.16 (m, 1H), 2.69 (d, 2H, J=20.8 Hz), 2.60-2.50 (m, 3H), 2.25 (m, 1H), 1.84 (m, 1H), 1.78-1.16 (m, 10H). FABMS m/z 775 M⁺, 776 (MH)⁺.

Conversion of 4 to Its Tri-sodium Salt. Compound 4 (5.2 mg, 0.0067 mmol) was dissolved in a solution of acetonitrile: H₂O (1.0 mL, v/v=1:1) and to this solution was added a NaHCO₃ solution (0.334 mL, 0.0201 mmol) and the resulting solution was lyophilized to provide the tri-sodium salt of 4 as white solid (5.6 mg, 100% yield).

General Procedure for Reductive Amination on Resin. To a suspension of 4-(4-formyl)-3-methoxyphenoxy)butyryl-NovaGel HL resin 13 (55 mg, 0.030 mmol; Novabiochem, Inc.) in dry 1,2-dichloroethane-trimethyl orthoformate (2:1, 1.2 mL) were added 1-naphthylmethylamine (0.044 mL, 0.30 mmol) and NaBH(OAc)₃ (64 mg, 0.30 mmol), and agitation was continued for 12 h at room temperature. The resin was washed successively with DMF, 10% i-Pr₂NEt/DMF and DMF to provide N-(1-naphthylmethylamino)-modified resin 14.

General Procedure for the Solid-Phase Synthesis of Protected Peptides on Resin. Protected peptide-resins were manually constructed by Fmoc-based solid-phase peptide synthesis. Trityl was employed for Asn side-chain protection. Fmoc deprotection was achieved by 20% piperidine in DMF (2×1 min, 1×20 min). Fmoc-amino acids were coupled by treatment with 5 equivalents of Fmoc-amino acid and coupling reagents [HATU/HOAt for Gly and AllylGly for 6 h; (DIPCDI)/HOBt for Asn(Trt) for 2 h; DIPCDI/HOAt for Ac₆c for 6 h] in DMF. The pTyr mimetic 10 was coupled using DIPCDI/HOAt in DMF for 2 days at 50° C.

[(2R)-3-(4-Dihydroxyphosphonomethyl)phenyl-2-(hydroxylcarbonylmethyl) propionyl]-Ac₆c-L-Asn-Gly-(1-naphthyl)methyl Amide (2). Protected peptide resin 16 (87 mg, 0.030 mmol) resulting from elaboration of modified resin 14 using the appropriate amino acids as described above under general procedures for solid-phase synthesis, was treated with TFA: H₂O (95:5, 10 mL) for 2 h at room temperature. After removal of resin by filtration, the filtrate was concentrated and purified by preparative (linear gradient from 30 to 40% B in A over 30 min) to provide 2 as colorless powder (10 mg, 45% yield based on resin substitution). ¹H-NMR (400 MHz, DMSO-d₆) δ 1.00-1.29 (m, 2H), 1.29-1.63 (m, 6H), 1.82 (m, 2H), 2.04 (dd, J=16.6 and 3.9 Hz, 1H), 2.43-2.65 (m, 4H), 2.90 (d, J=21.3 Hz, 2H), 2.96 (m, 1H), 3.11 (m, 1H), 3.68 (dd, J=16.9 and 5.7 Hz, 1H), 3.83 (dd, J=16.9 and 6.4 Hz, 1H), 4.34 (dt, J=7.4 and 5.7 Hz, 2H), 4.73 (m, 2H), 6.91 (br, 1H), 7.09-7.19 (m, 4H), 7.42 (m, 3H), 7.54 (m, 2H), 7.73 (d, J=7.4 Hz, 1H); 7.81 (m, 1H), 7.90-8.00 (m, 2H), 8.06 (m, 1H), 8.11 (t, J=5.7 Hz, 1H), 8.28 (s, 1H). FABMS m/z 736 [(M-H)⁻].

H—Ac₆c-L-Asn-D-AllylGly-(1-naphthyl)methyl amide (9b). Protected peptide resin 15c (429 mg, 0.15 mmol), resulting from elaboration of modified resin 14 using the appropriate amino acids as described above under general procedures for solid-phase synthesis, was treated with TFA: H₂P (95:5, 10 mL) for 2 h at room temperature. After filtration, the filtrate was concentrated and neutralized with saturated NaHCO₃ solution. The whole was extracted with EtOAc, and the extract was washed with brine, and dried over Na₂SO₄. Concentration followed by silica gel flash chromatography using EtOAc:MeOH (8:2) provided 9b (74 mg, quant.) as colorless solid. ¹H-NMR (400 MHz, DMSO-d₆) δ 1.13 (m, 1H), 1.27 (m, 2H), 1.36-1.57 (m, 5H), 1.66 (m, 2H), 2.34 (m, 1H), 2.40-2.58 (m, 3H), 4.31 (m, 1H), 4.45 (m, 1H), 4.74 (d, J=5.8 Hz, 2H), 4.98 (d, J=10.2 Hz, 1H), 5.04 (d, J=17.2 and 1.6 Hz, 1H), 5.69 (m, 1H), 6.95 (br, 1H), 7.45 (m, 3H), 7.54 (m, 2H), 7.84 (dd, J=7.0 and 2.3 Hz, 1H), 7.92 (m, 2H), 8.06 (m, 1H), 8.48 (m, 2H). FABMS m/z 494 (MH⁺).

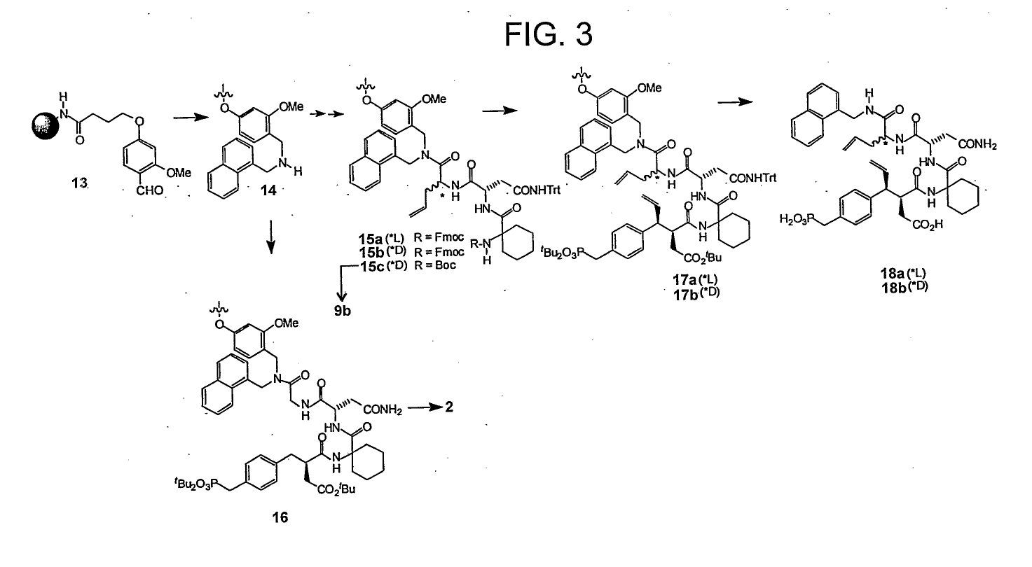

Preparation of 15a, 15b, 17a, 17b, 18a, and 18b. As illustrated in FIG. 3, elaboration of resin 14 by coupling with either N-Fmoc L-allylglycine or N-Fmoc D-allylglycine gave resins 15a and 15b, respectively. Coupling of Asn and Ac₆c residues followed by pTyr mimetic 10 yielded resins 17a and 17b, respectively. HPLC analysis of small samples of cleaved resin provided single major peaks that gave MALDI mass spectra consistent with the structure of peptides 18a and 18b, respectively. This indicated that the coupling of pTyr mimetic 10 had been achieved satisfactorily. Peptides 18a and 18b may be converted to compounds 3 and 4, respectively, by an RCM reaction.

Preparation of 20: 1-Chloro-1-cyanocyclohexane (J. Org. Chem. 1968, 33, 2211-2214). To a solution of phosphorus pentachloride (66.8 g, 0.32 mol) and pyridine (34 mL, 0.42 mol) in chloroform (360 mL, HPLC grade), was added cycanocyclohexane 19 (25 mL, 0.21 mol), the mixture was heated to reflux overnight. The reaction mixture was cooled to r.t., poured into crashed ice carefully, and the aqueous phase was extracted by ether (200 mL×2), the combined organic phase was washed by water (200 mL×3), sat. NaHCO₃ (100 mL), brine (100 mL), dried over anhydrous Na₂SO₄, after concentrated, the residue oil was distilled to afford 20 as a colorless liquid 28.6 in 95% yield. 73° C./4 mmHg; ¹H NMR (CDCl₃) δ 2.33 (2H, m), 2.00 (2H, m), 1.83 (2H, m), 1.75-1.61 (3H, m), 1.40 (1H, m); ¹³C NMR (CDCl₃) δ 119.5, 57.3, 40.2, 24.0, 23.2.

Preparation of 21 (Org. Lett. 2004, 6, 501-503): To a solution of 20 (5.0 g, 34.8 mmol) and allyl bromide (3.25 mL, 38.3 mmol) in dry THF (150 mL) at −78° C., was added n-BuLi (2.5 M, 15.3 mL, 38.3 mmol); the mixture was stirred at 78° C. for 2 hours before sat. NH₄Cl (30 mL) was added. The mixture was extracted with ethyl acetate, washed with brine, dried over anhydrous Na₂SO₄, purified by column (silica gel, hexane and ethyl acetate) to afford 21 as a pale yellow oil 3.8 g in 76% yield. ¹H NMR (CDCl₃) δ 5.90 (1H, m), 5.20 (2H, m), 2.30 (2H, m), 2.00 (2H, m), 1.85-1.50 (6H, m), 1.25 (2H, m); ¹³C NMR (CDCl₃) δ131.9, 123.0, 119.6, 44.6, 35.3, 25.3, 22.9.

Preparation of 22. To a solution of C (3.80 g, 25.5 mmol) in dry ether (200 mL) at 0° C., was added LiAlH₄ (2.0 g, 52.6 mmol) carefully, the mixture was stirred at r.t. overnight. The reaction mixture was cooled to 0° C. before water (5.0 mL) was added carefully, stirred vigorously until the color turned to white, dried over anhydrous Na₂SO₄, filtered, and the filtrate was concentrated to a colorless oil (free amine, 3.20 g) which was used directly for the next step. To a pre-prepared actived ester solution [Boc-Asn-OH (1.16 g, 5.0 mmol), HOBT (0.70 g, 5.0 mmol), EDCI (1.00 g, 5.0 mmol) and DIPEA (1.75 mL, 10 mmol) was mixture in dry DCM (25 mL) and stirred at r.t. for 15 mins], free amino (0.75 g, 5.0 mmol) was added, the mixture was stirred at r.t. overnight. Ethyl acetate (200 mL) was added, washed by water and brine, dried over anhydrous Na₂SO₄, purified by column (silical gel, hexane and ethyl acetate) to afford 22 as a colorless solid 200 mg in 11% yield over 2 steps. ¹H NMR (CDCl₃) δ 6.94 (1H, s), 6.29 (1H, s), 6.20 (1H, d, J=6.4 Hz), 5.84 (1H, m), 5.64 (1H, s), 5.10 (2H, m), 4.45 (1H, m), 3.17 (2H, m), 2.94 (1H, dd, J=15.2, 3.6 Hz), 2.55 (1H, dd, J=15.4, 6.2 Hz), 2.03 (2H, m), 150-1.20 (19H, m); FAB-MS (+VE) m/z 368.4 (MH⁺).

Preparation of 23. To a solution of 22 (47 mg, 0.128 mmol) in DCM (1.50 mL), was added TES (0.32 mL) and TFA (0.80 mL), the mixture was stirred at r.t. for 1.5 hrs. the solvent was removed in vacuo and the residue was used directly. To a solution of pre-prepared actived ester of 25 [a mixture of 25 (72 mg, 0.116 mmol), HOAT (19 mg, 0.140 mmol), EDCI (28 mg, 0.140 mmol) and DIPEA (91 μL, 0.522 mmol) in DMF (4.0 mL) was stirred at r.t. for 15 mins], was added a solution of free amine in DMF (2.0 mL), the mixture was stirred at r.t. for 18 hrs. Ethyl acetate (100 mL) was added to the reaction mixture, washed by water and brine, dried over anhydrous Na₂SO₄, purified by column (silica gel, chloroform and methanol) to afford 23 as a colorless solid 60 mg in 60% yield. ¹H NMR (CDCl₃) δ 7.27-7.16 (5H, m), 7.10 (1H, m), 6.67 (1H, s), 6.00 (1H, s), 6.00-5.80 (2H, m), 5.40 (1H, s), 5.12-5.05 (4H, m), 4.55 (1H, m), 3.50 (1H, t, J=14.0 Hz), 3.25 (1H, dd, J=13.2, 6.8 Hz), 3.10-2.95 (4H, m), 2.83-2.70 (2H, m), 2.64 (2H, m), 2.08 (2H, m), 1.90-1.10 (47H, m); FAB-MS (+VE) m/z 871.6 (MH⁺).

Preparation of 24. The solution of 23 (60 mg, 0.069 mmol) in dichloroethane (35 mL) was degassed for 5 mins under Ar, Grubbs's catalyst II (30 mg) was added, and the mixture was refluxed for 24 hrs. The solvent was removed in vacuo, the residue was purified by column (silica gel, hexane and ethyl acetate); the crude product was treated with TFA (9.5 mL), H₂O (0.5 mL) and TES (0.50 mL) at r.t. for 2 hrs, the final product was purified by HPLC, after lyophilized, 24 was got as a white power, 8 mg, 17% yield over 2 steps. ¹H NMR (CDCl₃) δ 8.36 (2H, m), 7.55 (1H, s) 7.30 (2H, AB, J_{AB}=8.0 Hz), 7.19 (2H, AB, J_{AB}=8.0 Hz), 7.16 (1H, s), 6.59 (1H, m), 5.80 (1H, dd, J=15.2, 9.6 Hz), 5.66 (1H, m), 4.28 (1H, m), 4.13 (1H, m), 3.60 (2H, m) 3.16 (1H, m), 2.90 (2H, d; J=21.2 Hz), 2.77 (2H, m), 2.36 (1H, dd, J=16.0, 4.8 Hz), 2.20 (1H, m), 2.07-1.94 (2H, m), 1.90-1.40 (20H, m); FAB-MS (−VE) m/z 673.4 (M-H)⁻.

### EXAMPLE 2

This example illustrates a property of compounds in accordance with an embodiment of the invention.

Biosensor Analysis: Binding experiments were performed on a Biacore S51 instrument (Biacore Inc., Piscataway N.J.). All Biotinylated Grb2 SH2 domain proteins (b-Grb2) were expressed and purified (Protein Expression Laboratory and The Protein Chemistry Laboratory, SAIC—Frederick). The b-Grb2 was immobilized onto carboxymethyl 5′ dextran surface (CM5 sensor chip, Biacore Inc.) by amine coupling. The lyophilized b-Grb2 was reconstituted in fifty percent DMSO in H₂O to make a stock solution of 1 mg/mL and stored at −80° C. A 1: 12.5 dilution of b-Grb2 was used for immobilization, by dilution in acetate buffer pH-5.0, with 5% DMSO. 1×PBS (phosphate buffered saline, pH 7.4) was used as the running buffer.

An immobilization wizard was used to optimize the immobilization target. For b-Grb2, 2500-5000 resonance units (RU) of protein were captured on the CM5 sensor chip. Small molecules were serially diluted in running buffer to the concentrations (1.25 nM-1500 nM) as indicated in each sensorgram and injected at 25° C. at a flow rate of 30 μL/min for 2 minutes. Varying concentrations of small molecules were injected in increasing concentrations, and every injection was performed in duplicate within each experiment. In order to subtract background noise from each data set, all samples were also run over an unmodified reference surface and random injections of running buffer were performed throughout every experiment (“double referencing”). Data were fit to a simple 1:1 interaction model, using the global data analysis program CLAMP; Myszka et al., Trends Biochem. Sci. 1998, 23, 149-150.

Effect of Macrocyclization on Grb2 SH2 Domain Binding Affinity. Using plasmon resonance techniques, steady state K_{D} values were obtained for direct binding of peptides 2, 3 and 4 to chip-bound Grb2 SH2 domain protein. Relative to the open-chain compound 2 (5610±75.0 nM), macrocycles 3 (K_{D}=22.7 L 0.455 nM) and 4 (54.9±0.945 nM) provided two orders of magnitude potency enhancements.

Grb2 SH2 Domain-Binding Affinity. Binding affinity of 24 to Grb2 SH2 domain protein was determined. Complex binding kinetics were observed with Kd values of 9.3±0.1 nM and 21.3±0.5 μM, respectively.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A compound of formula (I): 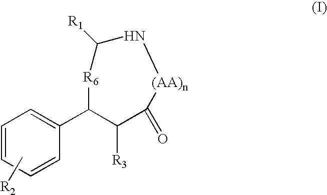
wherein R₁ is a lipophile; R₂, in combination with the phenyl ring, is a phenylphosphate mimic group or a protected phenylphosphate mimic group; R₃ is hydrogen, azido, amino, oxalylamino, carboxy C₁-C₆ alkyl, C₁-C₆ alkoxycarbonyl C₁-C₆ alkyl, aminocarbonyl C₁-C₆ alkyl, or C₁-C₆ alkyl carbonylamino; wherein the alkyl portion of R₃ may be optionally substituted with a substituent selected from the group consisting of halo, hydroxy, carboxyl, amino, amino C₁-C₆ alkyl, C₁-C₆ alkyl, C₁-C₆ alkoxy, and keto, and any combination thereof; R₆ is a linker; AA is an amino acid or fragment thereof; and n is 1 to 6; or a pharmaceutically acceptable salt, stereoisomer, solvate, or hydrate thereof.

2. The compound or a pharmaceutically acceptable salt, stereoisomer, solvate, or hydrate of claim 1, wherein n is 2 or 3.

3. The compound of claim 1 having the formula (Ia): 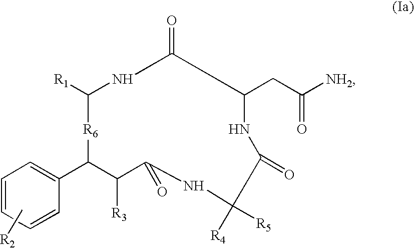
wherein R₁ is a lipophile; R₂, in combination with the phenyl ring, is a phenylphosphate mimic group or a protected phenylphosphate mimic group; R₃ is hydrogen, azido, amino, oxalylamino, carboxy C₁-C₆ alkyl, C₁-C₆ alkoxycarbonyl C₁-C₆ alkyl, aminocarbonyl C₁-C₆ alkyl, or C₁-C₆ alkyl carbonylamino; wherein the alkyl portion of R₃ may be optionally substituted with a substituent selected from the group consisting of halo, hydroxy, carboxyl, amino, amino C₁-C₆ alkyl, C₁-C₆ alkyl, C₁-C₆ alkoxy, and keto, and any combination thereof; R₄ and R₅, independently, are hydrogen, C₁-C₆ alkyl, C₄-C₈ cycloalkyl, or heterocyclyl, or R₄ and R₅ together form a C₄-C₈ cycloalkyl or heterocyclyl.
R₆ is a linker; AA is an amino acid or fragment thereof; and n is 1 to 6; or a pharmaceutically acceptable salt, stereoisomer, solvate, or hydrate thereof.

4. The compound or a pharmaceutically acceptable salt, stereoisomer, solvate, or hydrate of claim 1, wherein R₁ is selected from the group consisting of alkyl, alkoxy, alkenyl, alkynyl, aryl, aryloxy, aryl alkoxy, alkylaryl, alkyloxy aryl, arylalkyl, alkylamino, arylalkylamino, alkenylamino, arylamino, aryloxy alkyl, heterocyclyl, heterocyclyloxy, aryl heterocyclyl alkyl, heterocyclyl alkyl, heterocyclyl alkoxy, aryl heterocyclyl, aryl heterocyclyloxy, alkyl arylalkyl, alkoxy arylalkyl, and alkoxy arylalkoxy, and any combination thereof, optionally substituted or in combination with one or more groups such as alkyl, keto, ester, amino, aminocarbonyl, ureido, hydroxyl, thiol, cyano, alkoxy, and halo.

5. The compound or a pharmaceutically acceptable salt, stereoisomer, solvate, or hydrate of claim 1, wherein R₁ is C₁-C₆ alkyl carbonyl, C₆-C₁₄ aryl carbonyl, C₆-C₁₄ aryl C₁-C₆ alkyl carbonyl, C₆-C₁₄ aryl C₁-C₆ alkylamino carbonyl, C₆-C₁₄ aryl C₁-C₆ alkyl, C₆-C₁₄ aryl heterocyclyl C₁-C₆ alkyl, C₆-C₁₄ aryl heterocyclyl C₁-C₆ alkyl carbonyl, C₁-C₆ alkylaminocarbonyl, C₂-C₆ alkenylaminocarbonyl, C₆-C₁₄ arylaminocarbonyl, C₁-C₆ alkoxy C₁-C₆ alkyl, C₁-C₆ alkoxy C₁-C₆ alkyl carbonyl, C₆-C₁₄ aryloxy C₁-C₆ alkyl, C₆-C₁₄ aryloxy C₁-C₆ alkyl carbonyl, C₆-C₁₄ aryl C₁-C₆ alkoxy C₁-C₆ alkyl, or C₆-C₁₄ aryl C₁-C₆ alkoxy C₁-C₆ alkyl carbonyl, wherein the aryl portion is unsubstituted or substituted with a substituent selected from the group consisting of halo, hydroxy, carboxyl, amino, aminoalkyl, C₁-C₆ alkyl, C₁-C₆ alkoxy, and keto, and any combination thereof.

6. The compound or a pharmaceutically acceptable salt, stereoisomer, solvate, or hydrate of claim 5, wherein R₁ is C₁-C₆ alkyl carbonyl, C₆-C₁₄ aryl carbonyl, C₆-C₁₄ aryl C₁-C₆ alkyl carbonyl, C₆-C₁₄ aryl C₁-C₆ alkylamino carbonyl, C₆-C₁₄ aryl heterocyclyl C₁-C₆ alkyl carbonyl, C₁-C₆ alkylaminocarbonyl, C₂-C₆ alkenyl aminocarbonyl, C₆-C₁₄ arylamino carbonyl, C₁-C₆ alkoxy C₁-C₆ alkyl carbonyl, C₆-C₁₄ aryloxy C₁-C₆ alkyl carbonyl, or C₆-C₁₄ aryl C₁-C₆ alkoxy C₁-C₆ alkyl carbonyl, wherein the aryl portion is unsubstituted or substituted with a substituent selected from the group consisting of halo, hydroxy, carboxyl, amino, aminoalkyl, C₁-C₆ alkyl, C₁-C₆ alkoxy, and keto, and any combination thereof;

7. The compound or a pharmaceutically acceptable salt, stereoisomer, solvate, or hydrate of claim 1, wherein R₂ is hydroxyl, carboxyl, formyl, carboxy C₁-C₆ alkyl, carboxy C₁-C₆ alkoxy, dicarboxy C₁-C₆ alkyl, dicarboxy C₁-C₆ alkyloxy, dicarboxyhalo C₁-C₆ alkyl, dicarboxyhalo C₁-C₆ alkyloxy, phosphono, phosphono C₁-C₆ alkyl, phosphonohalo C₁-C₆ alkyl, phosphoryl, phosphoryl C₁-C₆ alkyl, and phosphoryl C₁-C₆ alkoxy, carboxy C₁-C₆ alkylamino, oxalylamino, RSO₂NH— wherein R can be C₁-C₆ alkyl, halo C₁-C₆ alkyl, C₆-C₁₄ aryl, C₆-C₁₄ aryl C₁-C₆ alkyl, or trifluoro C₁-C₆ alkyl, C₆-C₁₄ aryl C₁-C₆ alkyl, phosphino C₁-C₆ alkyl, C₁-C₆ alkyl phosphino C₁-C₆ alkyl, C₆-C₁₄ aryl, and C₆-C₁₄ aryl C₁-C₆ alkyl, wherein the alkyl and alkoxy portions of R₂ may be optionally substituted with a substituent selected from the group consisting of halo, hydroxy, carboxyl, amino, aminoalkyl, C₁-C₆ alkyl, C₁-C₆ alkoxy, and keto, and any combination thereof.

8. The compound or a pharmaceutically acceptable salt, stereoisomer, solvate, or hydrate of claim 7, wherein R₂ is hydroxyl, carboxyl, formyl, carboxy C₁-C₆ alkyl, carboxy C₁-C₆ alkoxy, dicarboxy C₁-C₆ alkyl, dicarboxy C₁-C₆ alkyloxy, dicarboxyhalo C₁-C₆ alkyl, dicarboxyhalo C₁-C₆ allyloxy, phosphono, phosphono C₁-C₆ alkyl, phosphonohalo C₁-C₆ alkyl, phosphoryl, phosphoryl C₁-C₆ alkyl, or phosphoryl C₁-C₆ alkoxy, wherein the alkyl and alkoxy portions may be optionally substituted with a substituent selected from the group consisting of halo, hydroxy, carboxyl, amino, amino C₁-C₆ alkyl, C₁-C₆ alkyl, C₁-C₆ alkoxy, and keto, and any combination thereof.

9. The compound or a pharmaceutically acceptable salt, stereoisomer, solvate, or hydrate of claim 3, wherein R₄ and R₅ together form a C₄-C₈ cycloalkyl.

10. The compound or a pharmaceutically acceptable sat, stereoisomer, solvate or hydrate of claim 9, wherein R₄ and R₅ together form cyclohexyl.

11. The compound or a pharmaceutically acceptable salt, stereoisomer, solvate, or hydrate of claim 1, wherein R₆ is a group having 1-6 carbon atoms, which may be optionally have a substituent selected from the group consisting of halo, hydroxy, carboxyl, amino, aminoalkyl, C₁-C₆ alkyl, C₁-C₆ alkoxy, and keto, and any combination thereof.

12. The compound or a pharmaceutically acceptable salt stereoisomer, solvate, or hydrate of claim 11, wherein R₆ is a C₂-C₆ alkenylenyl or C₂-C₆ alkynylenyl group, which may optionally have a substituent selected from the group consisting of halo, hydroxy, carboxyl, amino, aminoalkyl, C₁-C₆ alkyl, C₁-C₆ alkoxy, and keto, and any combination thereof.

13. The compound or a pharmaceutically acceptable salt, stereoisomer, solvate or hydrate of claim 12 wherein R₆ is a C₂-C₆ alkenylenyl.

14. The compound pharmaceutically acceptable salt stereoisomer, solvate, or hydrate of claim 3 having the formula:

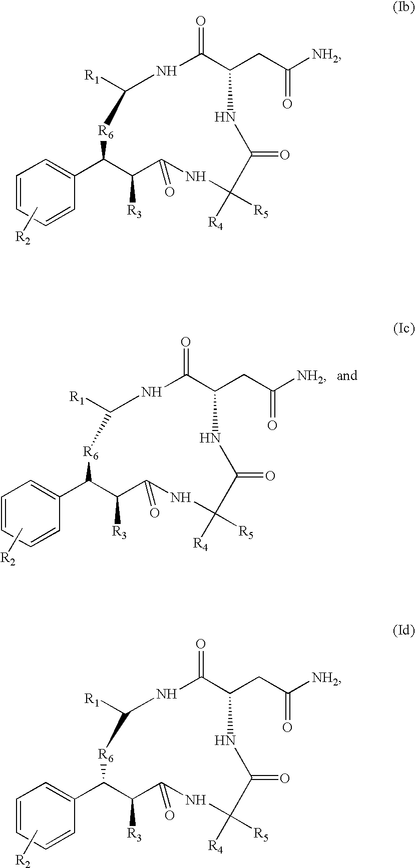

15. The compound or a pharmaceutically acceptable salt, stereoisomer, solvate, or hydrate of claim 14, wherein R₁ is C₁-C₆ alkyl carbonyl, C₆-C₁₄ aryl carbonyl, C₆-C₁₄ aryl C₁-C₆ alkyl carbonyl, C₆-C₁₄ aryl C₁-C₆ alkylamino carbonyl, C₆-C₁₄ aryl heterocyclyl C₁-C₆ alkyl carbonyl, C₁-C₆ alkylaminocarbonyl, C₂-C₆ alkenylaminocarbonyl, C₆-C₁₄ arylamino carbonyl, C₁-C₆ alkoxy C₁-C₆ alkyl carbonyl, C₆-C₁₄ aryloxy C₁-C₆ alkyl carbonyl, or C₆-C₁₄ aryl C₁-C₆ alkoxy C₁-C₆ alkyl carbonyl, wherein the aryl portion is unsubstituted or substituted with a substituent selected from the group consisting of halo, hydroxy, carboxyl, amino, aminoalkyl, C₁-C₆ alkyl, C₁-C₆ alkoxy, and keto, and any combination thereof;
R₂ is hydroxyl, carboxyl, formyl, carboxy C₁-C₆ alkyl, carboxy C₁-C₆ alkoxy, dicarboxy C₁-C₆ alkyl, dicarboxy C₁-C₆ alkyloxy, dicarboxyhalo C₁-C₆ alkyl, dicarboxyhalo C₁-C₆ alkyloxy, phosphono, phosphono C₁-C₆ alkyl, phosphonohalo C₁-C₆ alkyl, phosphoryl, phosphoryl C₁-C₆ alkyl, or phosphoryl C₁-C₆ alkoxy, wherein the alkyl and alkoxy portions may be optionally substituted with a substituent selected from the group consisting of halo, hydroxy, carboxyl, amino, amino C₁-C₆ alkyl, C₁-C₆ alkyl, C₁-C₆ alkoxy, and keto, and any combination thereof;
R₃ is hydrogen, azido, amino, oxalylamino, carboxy C₁-C₆ alkyl, C₁-C₆ alkoxycarbonyl C₁-C₆ alkyl, aminocarbonyl C₁-C₆ alkyl, or C₁-C₆ alkylcarbonyl amino; wherein the alkyl portion of R₃ may be optionally substituted with a substituent selected from the group consisting of halo, hydroxy, carboxyl, amino, amino C₁-C₆ alkyl, C₁-C₆ alkyl, C₁-C₆ alkoxy, and keto, and any combination thereof;
R₄ and R₅, independently, are hydrogen, C₁-C₆ alkyl, cycloalkyl, heterocyclyl, or together form cycloalkyl or heterocyclyl, wherein the cycloalkyl is a C₃-C₇ cycloalkyl, and the heterocyclyl is a 3-7 membered ring with at least one of N, O, and S; and
R₆ is a C₂-C₆ alkenylenyl or C₂-C₆ alkynylenyl group, which may be optionally substituted with a substituent selected from the group consisting of halo, hydroxy, carboxyl, amino, aminoalkyl, C₁-C₆ alkyl, C₁-C₆ alkoxy, and keto, and any combination thereof.

16. The compound or a pharmaceutically acceptable salt, stereoisomer, solvate, or hydrate of claim 15, wherein R₆ is C₂-C₆ alkenylenyl, which may be optionally substituted with a substituent selected from the group consisting of halo, hydroxy, carboxyl, amino, aminoalkyl, C₁-C₆ alkyl, C₁-C₆ alkoxy, and keto, and any combination thereof.

17. The compound or a pharmaceutically acceptable salt, stereoisomer, solvate, or hydrate of claim 16, wherein R₆ is C₂-C₄ alkenylenyl.

18. The compound or a pharmaceutically acceptable salt, stereoisomer, solvate, or hydrate of claim 1, wherein R₁ is C₆-C₁₄ aryl C₁-C₆ alkylamino carbonyl.

19. The compound or a pharmaceutically acceptable salt, stereoisomer, solvate, or hydrate of claim 18, wherein R₁ is C₁₀ aryl C₁-C₆ alkylaminocarbonyl.

20. The compound or a pharmaceutically-acceptable salt, stereoisomer, solvate, or hydrate of claim 19, wherein R₁ is naphthylmethylaminocarbonyl.

21. The compound or a pharmaceutically acceptable salt, stereoisomer, solvate, or hydrate of claim 1, wherein R₂ is phosphono C₁-C₆ alkyl, optionally substituted with a substituent selected from the group consisting of halo, hydroxy, carboxyl, amino, aminoalkyl, alkyl, alkoxy, and keto, and any combination thereof.

22. The compound or a pharmaceutically acceptable salt stereoisomer, solvate, or hydrate of claim 1, wherein R₂ is phosphonomethyl.

23. The compound or a pharmaceutically acceptable salt, stereoisomer, solvate, or hydrate of claim 1, wherein R₃ is carboxy C₁-C₆ alkyl.

24. The compound or a pharmaceutically acceptable salt, stereoisomer, solvate, or hydrate of claim 1, wherein R₃ is carboxymethyl.

25. The compound or a pharmaceutically acceptable salt, stereoisomer, solvate, or hydrate or claim 1, wherein R₆ is allyl.

26. The compound or a pharmaceutically acceptable salt, stereoisomer, solvate, or hydrate of claim 1, wherein said amino acids (AA)ₙ are selected from the group consisting of glycine, alanine, valine, norvaline, leucine, iso-leucine, norleucine, α-amino n-decanoic acid, serine, homoserine, threonine, methionine, cysteine, S-acetylamino-methyl-cysteine, proline, trans-3- and trans-4-hydroxyproline, phenylalanine, tyrosine, 4-aminophenylalanine, 4-nitrophenylalanine, 4-chlorophenylalanine, 4-carboxyphenylalanine, β-phenylserine, β-hydroxyphenylalanine, phenylglycine, α-naphthylalanine, cyclohexylalanine, cyclohexylglycine, tryptophan, indoline-2-carboxylic acid, 1,2,3,4-tetrahydroisoquinoline-3-carboxylic acid, aspartic acid, asparagine, aminomalonic acid, aminomalonic acid monoamide, glutamic acid, glutamine, histidine, arginine, lysine, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid and α,β-diaminopropionic acid, homophenylalanine, and α-tert-butylglycine, and any combination thereof.

27. The compound or a pharmaceutically acceptable salt, stereoisomer, solvate, or hydrate of claim 26, wherein said amino acids are asparagine and α-aminocyclohexane carboxylic acid.

28. A compound of the formula II: 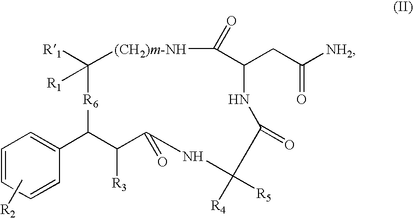
wherein R₁ and R₁′ are the same and are C₁-C₆ alkyl or R₁ and R₁′ together form a C₄-C₈ cycloalkyl; R₂, in combination with the phenyl ring, is a phenylphosphate mimic group or a protected phenylphosphate mimic group; R₃ is hydrogen, azido, amino, oxalylamino, carboxy C₁-C₆ alkyl, C₁-C₆ alkoxycarbonyl C₁-C₆ alkyl, aminocarbonyl C₁-C₆ alkyl, or C₁-C₆ alkyl carbonylamino; wherein the alkyl portion of R₃ may be optionally substituted with a substituent selected from the group consisting of halo, hydroxy, carboxyl, amino, amino C₁-C₆ alkyl, C₁-C₆ alkyl, C₁-C₆ alkoxy, and keto, and any combination thereof; R₄ and R₅, independently, are hydrogen, C₁-C₆ alkyl, C₄-C₈ cycloalkyl, or heterocyclyl, or R₄ and R₅ together form a C₄-C₈ cycloalkyl or heterocyclyl;
R₆ is a group having 1-6 carbon atoms, which may be optionally have a substituent selected from the group consisting of halo, hydroxy, carboxyl, amino, aminoalkyl, C₁-C₆ alkyl, C₁-C₆ alkoxy, and keto, and any combination thereof; and m is 1 or 2;
or a pharmaceutically acceptable salt, stereoisomer, solvate, or hydrate thereof.

29. The compound or a pharmaceutically acceptable salt stereoisomer, solvate, or hydrate of claim 28, wherein m is 1.

30. The compound or a pharmaceutically acceptable salt, stereoisomer, solvate, or hydrate of claim 28, wherein R₁ and R₁′ together form a C₄-C₈ cycloalkyl.

31. The compound or a pharmaceutically acceptable salt, stereoisomer, solvate, or hydrate of claim 30, wherein R₁ and R₁′ together form cyclohexyl.

32. The compound or a pharmaceutically acceptable salt, stereoisomer, solvate, or hydrate of claim 28, wherein R₂ is hydroxyl, carboxyl, formyl, carboxy C₁-C₆ alkyl, carboxy C₁-C₆ alkoxy, dicarboxy C₁-C₆ alkyl, dicarboxy C₁-C₆ alkyloxy, dicarboxyhalo C₁-C₆ alkyl, dicarboxyhalo C₁-C₆ alkyloxy, phosphono, phosphono C₁-C₆ alkyl, phosphonohalo C₁-C₆ alkyl, phosphoryl, phosphoryl C₁-C₆ alkyl, and phosphoryl C₁-C₆ alkoxy, carboxy C₁-C₆ alkylamino, oxalylamino, RSO₂NH— wherein R can be C₁-C₆ alkyl, halo C₁-C₆ alkyl, C₆-C₁₄ aryl, C₆-C₁₄ aryl C₁-C₆ alkyl, or trifluoro C₁-C₆ alkyl, C₆-C₁₄ aryl C₁-C₆ alkyl, phosphino C₁-C₆ alkyl, C₁-C₆ alkyl phosphino C₁-C₆ alkyl, C₆-C₁₄ aryl, and C₆-C₁₄ aryl C₁-C₆ alkyl, wherein the alkyl and alkoxy portions of R₂ may be optionally substituted with a substituent selected from the group consisting of halo, hydroxy, carboxyl, amino, aminoalkyl, C₁-C₆ alkyl, C₁-C₆ alkoxy, and keto, and any combination thereof.

33. The compound or a pharmaceutically acceptable salt, stereoisomer, solvate, or hydrate of claim 28, wherein R₄ and R₅ together form a C₄-C₈ cycloalkyl.

34. The compound or a pharmaceutically acceptable salt, stereoisomer, solvate, or hydrate of claim 33, wherein R₄ and R₅ together form cyclohexyl.

35. The compound pharmaceutically acceptable salt, stereoisomer, solvate, or hydrate of claim 28, wherein R₆ is a C₂-C₆ alkenylenyl or C₂-C₆ alkynylenyl group, which may optionally have a substituent selected from the group consisting of halo, hydroxy, carboxyl, amino, aminoalkyl, C₁-C₆ alkyl, C₁-C₆ alkoxy, and keto, and any combination thereof.

36. The compound or a pharmaceutically acceptable salt stereoisomer, solvate, or hydrate of claim 28, wherein R₃ is carboxy C₁-C₆ alkyl.

37. The compound or a pharmaceutically acceptable salt stereoisomer, solvate, or hydrate of claim 36, wherein R₃ is carboxy methyl.

38. The compound or a pharmaceutically acceptable salt, stereoisomer, solvate, or hydrate of claim 28, which has the formula IIa:

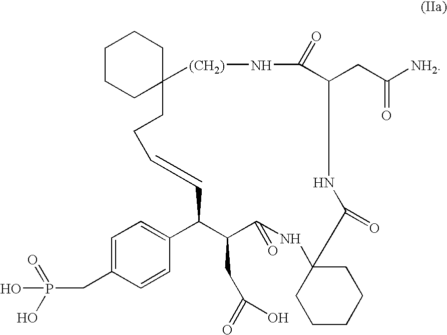

39. A pharmaceutical composition comprising a compound or a pharmaceutically acceptable salt, stereoisomer solvate, or hydrate of claim 1 and a pharmaceutically acceptable carrier.

40. A method for inhibiting an SH2 domain-containing protein from binding with a phosphoprotein comprising contacting the SH2 domain-containing protein with a compound or a pharmaceutically acceptable salt stereoisomer, solvate or hydrate of claim 1.

41. The method of claim 40, wherein the SH82 domain-containing protein is a Grb2 protein, Shp2 protein, or a STAT3 protein.

42. A method for treating a disease mediated by the binding of an SH2 domain-containing protein with a phosphoprotein, wherein the method comprises administering to a mammal afflicted with said disease an effective amount of a compound or a pharmaceutically acceptable salt, stereoisomer, solvate or hydrate of claim 1.

43. A pharmaceutical composition comprising a compound or a pharmaceutically acceptable salt, stereoisomer, solvate, or hydrate of claim 28 and a pharmaceutically acceptable carrier.

44. A method for inhibiting an SH2 domain-containing protein from binding with a phosphoprotein comprising contacting the SH2 domain-containing protein with a compound or a pharmaceutically acceptable salt, stereoisomer, solvate, or hydrate of claim 28.

45. A method for treating a disease mediated by the binding of an SH2 domain-containing protein with a phosphoprotein, wherein the method comprises administering to a mammal afflicted with said disease an effective amount of a compound or a pharmaceutically acceptable salt, stereoisomer, solvate, or hydrate of claim 28.
